SEQUENCE LISTING (107 sequences)

SEQ ID NO: 1 - protein, 118 aa, Mus musculus
QIQLVQSGPELKKPGETVKISCKASGYTFTNYGMNWVKQAPGKGLKWMGWINTYTGEPTYADDFKGRFAFSLETSASTAYLQINNLKNEDLATYFCATYGHYAWFAYWGQGTLVTVSA

SEQ ID NO: 2 - protein, 106 aa, Mus musculus
QIVLSQSPAILSASPGEKVTMTCRASSSVSYMHWYQQKSGSSPKPWIYATSNLASGVPARFSGSGSGTSYSLTISRVEAEDAATYFCQQWGTKPLTFGAGTKLELK

SEQ ID NO: 3 - protein, 118 aa, Mus musculus
QIQLVQSGPELKKPGETVKISCMASGYTFTTHGVTWVKQAPGQGLQRMGWINTYSGVPTYADDFKGRFVFSLETSANTAHLQINNLKNEDTATYFCARTNNYRYFDVWGTGTTVTVSS

SEQ ID NO: 4 - protein, 106 aa, Mus musculus
QIVLSQSPAILSSSPGEKVTMTCRASSSVSYMHWYQQKPGSSPKPWIYATSNLASGVPARFSGSGSGTSYFLTISRVEAEDAATYYCQQWNTDPPTFGGGTKLEIK

SEQ ID NO: 5 - protein, 119 aa, Mus musculus
QVTLKESGPGILQPSQTLSLTCSFSGFSLSTSGMGVGWIRQPSGKGLEWLAHIWWDDDKHCNPALKSRLTISKDTSSNQVFLKIASLDTADTATYYCTQMTMTREFAYWGQGTLVTVSA

SEQ ID NO: 6 - protein, 107 aa, Mus musculus
DIQMTQTISSLSASLGDRVTISCSAGQGISNYLNWYQQKPDGTVKLLIYYTSRLHSGVPSRFSGSGSGTDFSLTISNLEPEDIATYFCQQYSKFPYTFGGGTKLEIK

SEQ ID NO: 7 - protein, 119 aa, Mus musculus
DVQLQESGPGLVKPSQSLSLTCSVTGYSITGGYYWTWIRQFPGNKLEWMGYISYDGDNNYNPSLKNRISITRDTSKNQFFLNLNSVTTEDTATYYCAREGGRLRTMDYWGQGTSVTVSS

SEQ ID NO: 8 - protein, 106 aa, Mus musculus
QIVLTQSPAIMSASPGEKVTITCRASSSVNYMHWFQQKPGTSPKLWIYTTSNLASGVPARFSGSGSGTSYSLTISRMEAEDAATYYCQQRSSYPFTFGSGTKLEIK

SEQ ID NO: 9 - protein, 123 aa, Mus musculus
QVQLQQPGTELVKPGASVKLSCKTSGYTFTSYWMHWMKQRPGQGLEWLGHINPSTGGSNYNEKFKSKATLTVDKSSNTAYMQLSSLTSEDSAVYYCARPRGFYGYSYWYFDVWGSGTTVTVSS

SEQ ID NO: 10 - protein, 107 aa, Mus musculus
DIQMTQTTSSLSASLGDRVTISCSASQDINNYLNWYQHKPDGTIKVLIYFTSSLHSGVPSRFSGSGSGTEYSLTISNLEPEDIATYYCQQYSKLPPTFGSGTKLEIK

SEQ ID NO: 11 - protein, 121 aa, Mus musculus
QVTLRESGPGILQPSQTLSLTCSFSGFSLNTFGMGVGWIRQSSGKGLKWLAHIWWDDDKFYNPALKSRLTISKDTSKNQVFLKIANVDTTDTATYYCARIEGGLPYYYDYWGQGTPLIVSS

SEQ ID NO: 12 - protein, 107 aa, Mus musculus
DIVMTQSQKIMSTSVGDRISVTCKASQIVGTNVVWYQQKPGQSPKALIYSTSYRYSGVPDRFTGSRSGTDFTLTISNVQSEDLANYFCQQYNSYPFTFGSGTKLEIK

SEQ ID NO: 13 - protein, 122 aa, Mus musculus
QIQLVQSGPELKKPGETVKISCKASGYTFTAYGMSWVKQAPGKGLQWMGWINTYSGVPTYTDDFKGRFAFSLEASASTAFLQINNLKNEDTSTYFCARRAAVVAKYWYFDVWGTGTTVTVSS

SEQ ID NO: 14 - protein, 106 aa, Mus musculus
DIQMTQSPSSLFASLGGKVIITCKSSQDINKRISWYQHKPGKGPRLLIHSTSSLQPGIPLRFSGSGSGRDYSFSISNLEPEDIATYFCLQYDNLLTFGAGTKLELK

SEQ ID NO: 15 - protein, 120 aa, Mus musculus
QVQLQQSGPELVKPGASVRLSCKASGYTFTTYDINWVKQRPGLGLEWIGWIYPRDGYTKYNEKFKGRATLAVDTSSSTAYMELHSLTSEDSAVYFCTRGYYDNLYYFDYWGQGTTLTVSS

SEQ ID NO: 16 - protein, 108 aa, Mus musculus
EIVLTQSPTTVAASPGEKITITCSASSIISSNYFHWYQQRPGFSPKLLIYRTSNLASGVPARFSGSGSGTSYSLTIGTMEAEDVATYYCQQGRSVPLTFGAGTKLELK

SEQ ID NO: 17 - protein, 120 aa, Mus musculus
QVQLQQSGPELVKPGASVKLSCKASGYTFTSYDINWVKQRPGLGLEWIGWIYPRDGYTKYNEKFKGKATLAVDTSSSTAYMELHSLTSEDSAVYFCARGYYSNLYYFDYWGQGTTLTVSS

SEQ ID NO: 18 - protein, 108 aa, Mus musculus
EVVLTQSPPTMAASPGEKITITCSASSSISSNYLHWYQQKPGFSPKLLIYRTSNLASGVPARFSGSGSGTSYSLTIGTMEAEDVATYYCQQGRSIPLTFGAGTKLELK

SEQ ID NO: 19 - protein, 120 aa, Mus musculus
QVQLQQSGPELVKPGASVKLSCKASGYSFTTYDINWVKQRPGQGLEWIGWIYPRDGSTKYNEKFKGKATLTLDTSSNTAYMGLHSLTSEDSAVYFCARGYYDNLYYFDYWGQGTTLTVSS

SEQ ID NO: 20 - protein, 108 aa, Mus musculus
EIVLTQSPTTMAASPGEKITITCSASSSVSSNYLHWYQQKPGFSPKLLIYRTSNLASGVPARFSGSGSGTSYSLTIGTMEAEDVATYYCQQGRIVPLTFGAGTKLELK

SEQ ID NO: 21 - protein, 120 aa, Mus musculus
QVQLQQSGPELVKPGASVKLSCKASGYTFTSYDINWVKQRPGQGLEWIGWIYPRDGYTKYKEKFKGKATLTVDTSSSTAYMELHSLTSEDSAVYFCARGFYSNLYYFDYWGQGTTLTVSS

SEQ ID NO: 22 - protein, 108 aa, Mus musculus
EIVLTQSPTTMATSPGEKITITCSASSSISSNYLHWYQQKPGFSPKLLIYRTSNLASGVPDRFSGSGSGTSYSLTIGTMEAEDVATYYCQQGRSIPLTFGAGTKLELK

SEQ ID NO: 23 - protein, 120 aa, Mus musculus
QVQLQQSGPELVKPGASVKLSCKASGYTFTTYDINWVKQRPGLGLEWIGWIYPRDGYTKYNEKFKGKATLAVDTSSSTASMELHSLTSEDSAVYFCARGYYDNLYYFDNWGPGTTLTVSS

SEQ ID NO: 24 - protein, 108 aa, Mus musculus
ENVLTQSPTTVAASPGEKITITCSASSIISSNYLHWYQQKPGFSPKLLIYRTSNLASGVPARFSGSGSGTSYSLTIGTMEAEDAATYYCQQGRSVPLTFGAGTKLELK

SEQ ID NO: 25 - protein, 5 aa, Mus musculus
NYGMN

SEQ ID NO: 26 - protein, 17 aa, Mus musculus
WINTYTGEPTYADDFKG

SEQ ID NO: 27 - protein, 9 aa, Mus musculus
YGHYAWFAY

SEQ ID NO: 28 - protein, 10 aa, Mus musculus
RASSSVSYMH

SEQ ID NO: 29 - protein, 7 aa, Mus musculus
ATSNLAS

SEQ ID NO: 30 - protein, 9 aa, Mus musculus
QQWGTKPLT

SEQ ID NO: 31 - protein, 5 aa, Mus musculus
THGVT

SEQ ID NO: 32 - protein, 17 aa, Mus musculus
WINTYSGVPTYADDFKG

SEQ ID NO: 33 - protein, 9 aa, Mus musculus
TNNYRYFDV

SEQ ID NO: 34 - protein, 9 aa, Mus musculus
QQWNTDPPT

SEQ ID NO: 35 - protein, 7 aa, Mus musculus
TSGMGVG

SEQ ID NO: 36 - protein, 16 aa, Mus musculus
HIWWDDDKHCNPALKS

SEQ ID NO: 37 - protein, 9 aa, Mus musculus
MTMTREFAY

SEQ ID NO: 38 - protein, 11 aa, Mus musculus
SAGQGISNYLN

SEQ ID NO: 39 - protein, 7 aa, Mus musculus
YTSRLHS

SEQ ID NO: 40 - protein, 9 aa, Mus musculus
QQYSKFPYT

SEQ ID NO: 41 - protein, 6 aa, Mus musculus
GGYYWT

SEQ ID NO: 42 - protein, 16 aa, Mus musculus
YISYDGDNNYNPSLKN

SEQ ID NO: 43 - protein, 10 aa, Mus musculus
EGGRLRTMDY

SEQ ID NO: 44 - protein, 10 aa, Mus musculus
RASSSVNYMH

SEQ ID NO: 45 - protein, 7 aa, Mus musculus
TTSNLAS

SEQ ID NO: 46 - protein, 9 aa, Mus musculus
QQRSSYPFT

SEQ ID NO: 47 - protein, 5 aa, Mus musculus
SYWMH

SEQ ID NO: 48 - protein, 17 aa, Mus musculus
HINPSTGGSNYNEKFKS

SEQ ID NO: 49 - protein, 14 aa, Mus musculus
PRGFYGYSYWYFDV

SEQ ID NO: 50 - protein, 11 aa, Mus musculus
SASQDINNYLN

SEQ ID NO: 51 - protein, 7 aa, Mus musculus
FTSSLHS

SEQ ID NO: 52 - protein, 9 aa, Mus musculus
QQYSKLPPT

SEQ ID NO: 53 - protein, 7 aa, Mus musculus
TFGMGVG

SEQ ID NO: 54 - protein, 16 aa, Mus musculus
HIWWDDDKFYNPALKS

SEQ ID NO: 55 - protein, 11 aa, Mus musculus
IEGGLPYYYDY

SEQ ID NO: 56 - protein, 11 aa, Mus musculus
KASQIVGTNVV

SEQ ID NO: 57 - protein, 7 aa, Mus musculus
STSYRYS

SEQ ID NO: 58 - protein, 9 aa, Mus musculus
QQYNSYPFT

SEQ ID NO: 59 - protein, 5 aa, Mus musculus
AYGMS

SEQ ID NO: 60 - protein, 17 aa, Mus musculus
WINTYSGVPTYTDDFKG

SEQ ID NO: 61 - protein, 13 aa, Mus musculus
RAAVVAKYWYFDV

SEQ ID NO: 62 - protein, 11 aa, Mus musculus
KSSQDINKRIS

SEQ ID NO: 63 - protein, 7 aa, Mus musculus
STSSLQP

SEQ ID NO: 64 - protein, 8 aa, Mus musculus
LQYDNLLT

SEQ ID NO: 65 - protein, 5 aa, Mus musculus
TYDIN

SEQ ID NO: 66 - protein, 17 aa, Mus musculus
WIYPRDGYTKYNEKFKG

SEQ ID NO: 67 - protein, 11 aa, Mus musculus
GYYDNLYYFDY

SEQ ID NO: 68 - protein, 12 aa, Mus musculus
SASSIISSNYFH

SEQ ID NO: 69 - protein, 7 aa, Mus musculus
RTSNLAS

SEQ ID NO: 70 - protein, 9 aa, Mus musculus
QQGRSVPLT

SEQ ID NO: 71 - protein, 5 aa, Mus musculus
SYDIN

SEQ ID NO: 72 - protein, 11 aa, Mus musculus
GYYSNLYYFDY

SEQ ID NO: 73 - protein, 12 aa, Mus musculus
SASSSISSNYLH

SEQ ID NO: 74 - protein, 9 aa, Mus musculus
QQGRSIPLT

SEQ ID NO: 75 - protein, 17 aa, Mus musculus
WIYPRDGSTKYNEKFKG

SEQ ID NO: 76 - protein, 12 aa, Mus musculus
SASSSVSSNYLH

SEQ ID NO: 77 - protein, 9 aa, Mus musculus
QQGRIVPLT

SEQ ID NO: 78 - protein, 17 aa, Mus musculus
WIYPRDGYTKYKEKFKG

SEQ ID NO: 79 - protein, 11 aa, Mus musculus
GFYSNLYYFDY

SEQ ID NO: 80 - protein, 11 aa, Mus musculus
GYYDNLYYFDN

SEQ ID NO: 81 - protein, 12 aa, Mus musculus
SASSIISSNYLH

SEQ ID NO: 82 - protein, 118 aa, Synthetic construct
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARYGHYAWFAYWGQGTLVTVSS

SEQ ID NO: 83 - protein, 118 aa, Synthetic construct
QIQLVQSGSELKKPGASVKVSCKASGYTFTNYGMNWVRQAPGQGLEWMGWINTYTGEPTYADDFKGRFVFSLDTSVSTAYLQISSLKAEDTAVYYCATYGHYAWFAYWGQGTLVTVSS

SEQ ID NO: 84 - protein, 118 aa, Synthetic construct
QIQLVQSGSELKKPGASVKVSCKASGYTFTNYGMNWVRQAPGQGLEWMGWINTYTGEPTYADDFKGRFVFSLDTSVSTAYLQISSLKAEDTAVYFCATYGHYAWFAYWGQGTLVTVSS

SEQ ID NO: 85 - protein, 118 aa, Synthetic construct
QIQLVQSGSELKKPGASVKVSCKASGYTFTNYGMNWVRQAPGQGLKWMGWINTYTGEPTYADDFKGRFVFSLDTSVSTAYLQISSLKAEDTAVYFCATYGHYAWFAYWGQGTLVTVSS

SEQ ID NO: 86 - protein, 118 aa, Synthetic construct
QIQLVQSGSELKKPGASVKVSCKASGYTFTNYGMNWVKQAPGQGLKWMGWINTYTGEPTYADDFKGRFVFSLDTSVSTAYLQISSLKAEDTAVYFCATYGHYAWFAYWGQGTLVTVSS

SEQ ID NO: 87 - protein, 118 aa, synthetic construct
QVQLVQSGSELKKPGASVKISCKASGYTFTNYGMNWVRQAPGQGLEWMGWINTYTGEPTYADDFKGRFVFSLDTSVSTAYLQINNLKAEDTAVYFCATYGHYAWFAYWGQGTLVTVSS

SEQ ID NO: 88 - protein, 98 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCAR

SEQ ID NO: 89 - protein, 106 aa, synthetic construct
EIVLTQSPDFQSVTPKEKVTITCRASSSVSYMHWYQQKPDQSPKLLIKATSNLASGVPSRFSGSGSGTDFTLTINSLEAEDAATYYCQQWGTKPLTFGQGTKLEIK

SEQ ID NO: 90 - protein, 106 aa, synthetic construct
EIVLTQSPDFQSVTPKEKVTITCRASSSVSYMHWYQQKPDQSPKPWIKATSNLASGVPSRFSGSGSGTDFTLTINSLEAEDAATYYCQQWGTKPLTFGQGTKLEIK

SEQ ID NO: 91 - protein, 106 aa, synthetic construct
EIVLTQSPDFQSVTPKEKVTITCRASSSVSYMHWYQQKPDQSPKPWIYATSNLASGVPSRFSGSGSGTDFTLTINSLEAEDAATYFCQQWGTKPLTFGQGTKLEIK

SEQ ID NO: 92 - protein, 95 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 93 - protein, 120 aa, synthetic construct
QVQLVQSGAEVKKPGASVKVSCKASGYTFTTYDINWVRQAPGQGLEWMGWIYPRDGYTKYNEKFKGRVTMTTDTSTSTAYMELRSLRSDDTAVYYCARGYYDNLYYFDYWGQGTTVTVSS

SEQ ID NO: 94 - protein, 120 aa, synthetic construct
QVQLVQSGAEVKKPGASVKVSCKASGYTFTTYDINWVRQAPGQGLEWMGWIYPRDGYTKYNEKFKGRVTMTTDTSTSTAYMELRSLRSDDTAVYYCTRGYYDNLYYFDYWGQGTTVTVSS

SEQ ID NO: 95 - protein, 120 aa, synthetic construct
QVQLVQSGAEVKKPGASVKVSCKASGYTFTTYDINWVRQAPGQGLEWIGWIYPRDGYTKYNEKFKGRATMTTDTSTSTAYMELRSLRSDDTAVYFCTRGYYDNLYYFDYWGQGTTVTVSS

SEQ ID NO: 96 - protein, 120 aa, synthetic construct
QVQLVQSGAEVKKPGASVKVSCKASGYTFTTYDINWVRQAPGQGLEWIGWIYPRDGYTKYNEKFKGRATLAVDTSTSTAYMELRSLRSDDTAVYFCTRGYYDNLYYFDYWGQGTTVTVSS

SEQ ID NO: 97 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 98 - protein, 108 aa, synthetic construct
EIVLTQSPDFQSVTPKEKVTITCSASSIISSNYFHWYQQKPDQSPKLLIKRTSNLASGVPSRFSGSGSGTDFTLTINSLEAEDAATYYCQQGRSVPLTFGQGTKLEIK

SEQ ID NO: 99 - protein, 108 aa, synthetic construct
EIVLTQSPDFQSVTPKEKVTITCSASSIISSNYFHWYQQKPDQSPKLLIKRTSNLASGVPSRFSGSGSGTDYTLTINSLEAEDAATYYCQQGRSVPLTFGQGTKLEIK

SEQ ID NO: 100 - protein, 108 aa, synthetic construct
EIVLTQSPDFQSVTPKEKVTITCSASSIISSNYFHWYQQKPDQSPKLLIYRTSNLASGVPSRFSGSGSGTDYTLTINSLEAEDAATYYCQQGRSVPLTFGQGTKLEIK

SEQ ID NO: 101 - protein, 95 aa, Homo sapiens; identical to SEQ ID NO: 92

SEQ ID NO: 102 - protein, 122 aa, synthetic construct
QVQLVQSGSELKKPGASVKVSCKASGYTFTAYGMSWVKQAPGQGLQWMGWINTYSGVPTYTDDFKGRFVFSLDTSVSTAFLQISSLKAEDTAVYFCARRAAVVAKYWYFDVWGQGTTVTVSS

SEQ ID NO: 103 - protein, 98 aa, Homo sapiens; identical to SEQ ID NO: 88

SEQ ID NO: 104 - protein, 122 aa, synthetic construct
QVQLVQSGHEVKQPGASVKVSCKASGYSFTAYGMSWVKQAPGQGLQWMGWINTYSGVPTYTDDFKGRFVFSLDTSASTAFLQISSLKAEDMAMYFCARRAAVVAKYWYFDVWGQGTTVTVSS

SEQ ID NO: 105 - protein, 98 aa, Homo sapiens
QVQLVQSGHEVKQPGASVKVSCKASGYSFTTYGMNWVPQAPGQGLEWMGWFNTYTGNPTYAQGFTGRFVFSMDTSASTAYLQISSLKAEDMAMYYCAR

SEQ ID NO: 106 - protein, 106 aa, synthetic construct
DIQMTQSPSSLSASVGDRVTITCKSSQDINKRISWYQQKPGKAPKLLIHSTSSLQPGVPSRFSGSGSGRDFTFTISSLQPEDIATYFCLQYDNLLTFGQGTKLEIK

SEQ ID NO: 107 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

(19) United States
(12) Patent Application Publication
(10) Pub. No.: US 2026/0028422 A1
(43) Pub. Date: Jan. 29, 2026

(54) Anti-MerTK ANTIBODIES AND USES THEREOF

(71) Applicants: CONCEPT TO MEDICINE BIOTECH CO., LTD., Shanghai (CN); LEPU BIOPHARMA CO., LTD., Shanghai (CN)

(72) Inventors: Wenci GONG, Shanghai (CN); Zhijian CAI, Shanghai (CN); Qi WU, Shanghai (CN); Feifei CUI, Shanghai (CN); Lei FANG, Shanghai (CN)

(21) Appl. No.: 18/997,761

(22) PCT Filed: Jul. 28, 2023
(86) PCT No.: PCT/CN2023/109862

§ 371 Date: Jan. 22, 2025

(30) Foreign Application Priority Data
Jul. 28, 2022 (WO) PCT/CN2022/108736

(51) Int. Cl. C07K 16/40; A61K 40/31; C07K 14/725

(52) U.S. Cl. CPC C07K 16/40; A61K 40/31; C07K 14/7051; A61K 2239/13; A61K 2239/21; A61K 2239/22; C07K 2317/14; C07K 2317/24; C07K 2317/31; C07K 2317/565; C07K 2317/76

(57) ABSTRACT

This disclosure provides anti-MerTK antibodies, variants thereof and humanized versions. The newly disclosed antibodies exhibited high affinity to the MerTK protein and can be used to treat cancers, in particular solid tumors.

### BACKGROUND

Currently, most cancer immune-oncology therapies focus on modulating the activity of adaptive immune cells especially T cells by blocking inhibitory pathways including the checkpoint molecules or by redirecting them to fight against tumor through tumor associated antigen (TAA) engagement. However, data obtained from numerous investigations in mouse models of cancer and in humans with cancer offer compelling evidence that particular innate immune cell types including tumor associated macrophages (TAMs) and myeloid derived suppressive cells (MDSCs) predominantly function as extrinsic tumor-suppressor mechanisms to limit the clinical benefits of adaptive immune cell-based therapies. Targeting the innate immune system may complement the adaptive immune-oncology therapies to achieve enduring anti-tumor responses.

Macrophages of the innate immune system are a diverse collection of cell types with a wide range of functional roles in homeostatic and pathological conditions. M1 macrophages is a classically activated macrophages which behave pro-inflammatorily to clear intracellular pathogen. In contrast, M2 macrophages is an alternatively activated macrophages contributing to tissue repair and efferocytosis. Macrophages are professional phagocytes highly specialized in removal of dying or dead cells, and cellular debris which are generated abundantly under normal physiological condition. In addition, macrophages are detected to be extremely abundant in various types of solid tumors. Macrophages within the tumor microenvironment are known as tumor-associated macrophages (TAMs), which typically promote cancer cell initiation and proliferation, accelerate angiogenesis, and tame anti-tumor immunity to promote tumor progression and metastasis. Accumulating evidence indicate that TAMs may contribute to the relatively low response rate to T-cell based therapy. In solid tumors, uncontrolled tumor growth is often accompanied by increased cell death due to hypoxia and metabolic stress. To evade immune surveillance, tumors take advantage of the non-immunogenic nature of apoptosis. TAMs actively remove the dying tumor cells while sparing inflammatory cytokines production to avoid alerting the immune system.

MerTK (Mer proto-oncogene tyrosine-protein kinase) has been shown to play a role in clearance of apoptotic cells. MerTK is a member of the TYRO3/AXL/MER (TAM) receptor kinase family and encodes a transmembrane protein with two fibronectin type-III domains, two Ig-like C2-type (immunoglobulin-like) domains, and one tyrosine kinase domain. MerTK was observed with higher expression on M2 macrophages than M1 macrophages. MerTK facilitates removal of dying or damaged cells that display the “eat me” signal, phosphatidylserine (PtdSer), on the cell surface with the help of bridging molecule growth arrest specific 6 (Gas6) or protein S. MerTK-expressing macrophages engulf apoptotic cells via efferocytosis. In tumors, uncontrolled proliferation can cause increased apoptosis of cancer cells, but MerTK-dependent clearance of dying cells by TAMs might inhibit immune activation.

Blockade of MerTK results in accumulation of apoptotic cells within tumors and triggers a type I interferon response. Treatment of tumor-bearing mice with anti-MerTK antibody stimulates T cell activation and improves the efficacy of anti-PD-1 and anti-PD-L1 therapies. Thus, MerTK blockade increases tumor immunogenicity and potentiates anti-tumor immunity, and presents a therapeutic avenue to increase tumor immunogenicity and improve cancer immunotherapy.

### SUMMARY

The present disclosure, in various embodiments, provides antibodies and antigen-binding fragments specific to the human MerTK protein. One embodiment provides an antibody or antigen-binding fragment thereof which has specificity to the human Mer proto-oncogene tyrosine-protein kinase (MerTK) protein and comprises a heavy chain variable region (VH) comprising a VH CDR1, a VH CDR2 and a VH CDR3, and a light chain variable region (VL) comprising a VL CDR1, a VL CDR2, and a VL CDR3, wherein the VH CDR1, VH CDR2, VH CDR3, VL CDR1, VL CDR2 and VL CDR3 comprise, respectively, the amino acid sequences of SEQ ID NO: 25-30; SEQ ID NO: 59-64; SEQ ID NO: 65-70; SEQ ID NO: 31-33, 28-29, 34; SEQ ID NO: 35-40; SEQ ID NO: 41-46; SEQ ID NO: 47-52; SEQ ID NO: 53-58; SEQ ID NO: 71, 66, 72, 73, 69, 74; SEQ ID NO: 65, 75, 67, 76, 69, 77; SEQ ID NO: 71, 78-79, 73, 69, 74; or SEQ ID NO: 65-66, 80-81, 69-70.

In some embodiments, the VH CDR1 comprises the amino acid sequence of SEQ ID NO: 25; the VH CDR2 comprises an amino acid sequence of SEQ ID NO: 26; the VH CDR3 comprises the amino acid sequence of SEQ ID NO: 27; the VL CDR1 comprises the amino acid sequence of SEQ ID NO: 28; the VL CDR2 comprises the amino acid sequence of SEQ ID NO: 29; and the VL CDR3 comprises the amino acid sequence of SEQ ID NO: 30.

In some embodiments, the VH comprises an amino acid sequence selected from the group consisting of SEQ ID NO: 1, and 82-87, and the VL comprises an amino acid sequence selected from the group consisting of SEQ ID NO: 2 and 89-91.

In some embodiments, wherein the VH comprises the amino acid sequence of SEQ ID NO: 83, and the VL comprises the amino acid sequence of SEQ ID NO: 90. In some embodiments, the VH comprises the amino acid sequence of SEQ ID NO: 83, and the VL comprises the amino acid sequence of SEQ ID NO: 91.

In some embodiments, the VH CDR1 comprises the amino acid sequence of SEQ ID NO: 59; the VH CDR2 comprises an amino acid sequence of SEQ ID NO: 60; the VH CDR3 comprises the amino acid sequence of SEQ ID NO: 61; the VL CDR1 comprises the amino acid sequence of SEQ ID NO: 62; the VL CDR2 comprises the amino acid sequence of SEQ ID NO: 63; and the VL CDR3 comprises the amino acid sequence of SEQ ID NO: 64.

In some embodiments, the VH comprises an amino acid sequence selected from the group consisting of SEQ ID NO: 13, 102 and 104, and the VL comprises an amino acid sequence selected from the group consisting of SEQ ID NO: 14 and 106. In some embodiments, the VH comprises the amino acid sequence of SEQ ID NO: 102, and the VL comprises the amino acid sequence of SEQ ID NO: 106. In some embodiments, the VH comprises the amino acid sequence of SEQ ID NO: 104, and the VL comprises the amino acid sequence of SEQ ID NO: 106.

In some embodiments, the VH CDR1 comprises the amino acid sequence of SEQ ID NO: 65; the VH CDR2 comprises an amino acid sequence of SEQ ID NO: 66; the VH CDR3 comprises the amino acid sequence of SEQ ID NO: 67; the VL CDR1 comprises the amino acid sequence of SEQ ID NO: 68; the VL CDR2 comprises the amino acid sequence of SEQ ID NO: 69; and the VL CDR3 comprises the amino acid sequence of SEQ ID NO: 70.

In some embodiments, the VH comprises an amino acid sequence selected from the group consisting of SEQ ID NO: 15 and 93-96, and the VL comprises an amino acid sequence selected from the group consisting of SEQ ID NO: 16 and 98-100. In some embodiments, the VH comprises the amino acid sequence of SEQ ID NO: 95, and the VL comprises the amino acid sequence of SEQ ID NO: 99. In some embodiments, the VH comprises the amino acid sequence of SEQ ID NO: 96, and the VL comprises the amino acid sequence of SEQ ID NO: 99.

In some embodiments, the antibody or fragment thereof is a bivalent Fab antibody, or a fragment selected from the group consisting of F(ab′)2, F(ab)2, Fab′, Fab, Fv, and scFv.

Also provided, in one embodiment, is a multispecific antibody comprising an antigen-binding fragment of the present disclosure and one or more antibody or antigen-binding fragment having binding specificity to a target antigen that is not MerTK.

Yet further provided is a chimeric antigen receptor (CAR) comprising an antigen-binding fragment of the present disclosure, a transmembrane domain, a costimulatory domain, and a CD3ξ intracellular domain.

Also provided is one or more polynucleotide(s) encoding the antibody or antigen-binding fragment thereof or the CAR of the present disclosure. In some embodiments, the polynucleotide is one or more mRNA. In some embodiments, the mRNA is chemically modified.

Also provided, in one embodiment, is a method of treating cancer or an inflammatory condition in a patient in need thereof, comprising administering to the patient an effective amount of the antibody or antigen-binding fragment thereof or the CAR of the present disclosure. In some embodiments, the cancer is a solid tumor, such as bladder cancer, breast cancer, colorectal cancer, endometrial cancer, esophageal cancer, head and neck cancer, kidney cancer, leukemia, liver cancer, lung cancer, melanoma, pancreatic cancer, prostate cancer, gastric cancer, cervical cancer, uterus cancer, and thyroid cancer.

In some embodiments, the inflammatory condition is selected from the group consisting of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, and ulcerative colitis.

### BRIEF DESCRIPTION OF THE DRAWINGS

FIG. 1 shows that all tested anti-MerTK antibodies can efficiently bind to the human MerTK protein.

FIG. 2 shows that all the tested anti-MerTK antibodies can efficiently bind to the cyno MerTK protein.

FIG. 3 shows that most of the tested MerTK chimeric antibodies have higher maximum binding ability and binding potency to human MerTK expressed on CHO-K1 cells as compared to benchmark antibodies Ab2000-A7, M6, and h13B4.v16.

FIG. 4 shows that some of the tested MerTK chimeric antibodies have higher binding efficacy to human MerTK expressed on SK-MEL-5 cells as compared to benchmark antibodies Ab2000-A7, M6, and h13B4.v16.

FIG. 5 shows that all the antibodies can efficiently inhibit the binding of human MerTK to human Gas6 expressed on cells.

FIG. 6 shows that some of the tested MerTK antibodies have higher blocking efficiency in efferocytosis than benchmark antibody h13B4.v16.

FIG. 7 shows that all tested humanized antibodies have comparable binding efficacy to human MerTK protein to the chimeric antibody.

FIG. 8 shows that some of the tested humanized antibodies have comparable binding activity to human MerTK expressed on CHOK1 cells to their parental chimeric counterparts.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” includes aptamers, spiegeleisen, and diabodies. The term “antibody fragment” also includes any synthetic or genetically engineered protein that acts like an antibody by binding to a specific antigen to form a complex.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively.

The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000 Daltons. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

Antibodies disclosed herein can be from any animal origin including birds and mammals. Preferably, the antibodies are human, murine, donkey, rabbit, goat, guinea pig, camel, llama, horse, or chicken antibodies. In some embodiments, the variable region may be condricthoid in origin (e.g., from sharks).

As used herein, the term “recombinant” as it pertains to polypeptides or polynucleotides intends a form of the polypeptide or polynucleotide that does not exist naturally, a non-limiting example of which can be created by combining polynucleotides that would not normally occur together.

Hybridoma technology can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A nonlimiting example of a physiological condition is the temperature, ionic strength, pH and concentration of Mg2+ normally found in a cell.

### Anti-MerTK Antibodies

As demonstrated in the appended experimental examples, the instant inventors were able to generate anti-MerTK antibodies 10F7D9, 85H8D5, 216G3D6, 247E5A8, 252C12B10, 252H9D5, 254B4D9, 265F11B5, 276C2D1, 280C6A3, 293C2B7, and 300A5A3 (Table 1). Also importantly, many of these antibodies exhibited greater binding affinity than benchmark antibodies including M6, Ab2000-A7 and h13B4.v16 (as disclosed in WO2019084307A1, WO2016106221A1, and WO2020214995A1) to the human MerTK protein expressed on cells. Also, some of these antibodies exhibited higher ligand-binding blocking activities, as well as efferocytosis inhibition efficiency than the benchmark antibodies.

In accordance with one embodiment of the present disclosure, provided is an antibody or antigen-binding fragment thereof. In some embodiments, the antibody or antigen-binding fragment thereof has binding specificity to the human MerTK protein. In some embodiments, the antibody or antigen-binding fragment thereof includes a heavy chain variable region (VH) that includes a VH CDR1, a VH CDR2 and a VH CDR3, and a light chain variable region (VL) that includes a VL CDR1, a VL CDR2, and a VL CDR3.

In some embodiments, provided is an antibody or antigen-binding fragment that is derived from antibody 10F7D9. In some embodiments, the VH CDR1 includes the amino acid sequence of SEQ ID NO: 25; the VH CDR2 includes the amino acid sequence of SEQ ID NO: 26; the VH CDR3 includes the amino acid sequence of SEQ ID NO: 27; the VL CDR1 includes the amino acid sequence of SEQ ID NO: 28; the VL CDR2 includes the amino acid sequence of SEQ ID NO: 29; and the VL CDR3 includes an amino acid sequence selected from the group consisting SEQ ID NO: 30.

An example VH sequence includes an amino acid sequence selected from the group consisting of SEQ ID NO: 1, and 82-87. An example VL sequence includes an amino acid sequence selected from the group consisting of SEQ ID NO: 2 and 89-91.

In some embodiments, the VH includes an amino acid sequence of any one of SEQ ID NO: 1, and 82-87, or a sequence having at least 75%, 80%, 85%, 90%, 95% or 99% sequence identity to any one of SEQ ID NO: 1, and 82-87, while retaining the corresponding VH CDRs. In some embodiments, the VL includes an amino acid sequence of any one of SEQ ID NO: 2 and 89-91, or a sequence having at least 75%, 80%, 85%, 90%, 95% or 99% sequence identity to any one of SEQ ID NO: 2 and 89-91, while retaining the corresponding VL CDRs.

In some embodiments, the VH includes the amino acid sequence of SEQ ID NO: 83 and the VL includes an amino acid sequence of any one of SEQ ID NO: 2 and 89-91. In some embodiments, the VH includes the amino acid sequence of SEQ ID NO: 83 and the VL includes the amino acid sequence of SEQ ID NO: 90. In some embodiments, the VH includes the amino acid sequence of SEQ ID NO: 83 and the VL includes the amino acid sequence of SEQ ID NO: 91.

In some embodiments, the VH includes an amino acid sequence of any one of SEQ ID NO: 1, and 82-87 and the VL includes the amino acid sequence of SEQ ID NO: 90. In some embodiments, the VH includes an amino acid sequence of any one of SEQ ID NO: 82-87 and the VL includes the amino acid sequence of SEQ ID NO: 90. In some embodiments, the VH includes an amino acid sequence of any one of SEQ ID NO: 1, and 82-87 and the VL includes the amino acid sequence of SEQ ID NO: 91. In some embodiments, the VH includes an amino acid sequence of any one of SEQ ID NO: 82-87 and the VL includes the amino acid sequence of SEQ ID NO: 91.

Also provided, in some embodiments, are antibodies and antigen-binding fragments therefore that bind to the same epitope on MerTK as 10F7D9. Also provided, in some embodiments, are antibodies and antigen-binding fragments therefore that competes with 10F7D9 in binding to MerTK.

In some embodiments, provided is an antibody or antigen-binding fragment that is derived from antibody 85H8D5. In some embodiments, the VH CDR1 includes the amino acid sequence of SEQ ID NO: 31; the VH CDR2 includes the amino acid sequence of SEQ ID NO: 32; the VH CDR3 includes the amino acid sequence of SEQ ID NO: 33; the VL CDR1 includes the amino acid sequence of SEQ ID NO: 28; the VL CDR2 includes the amino acid sequence of SEQ ID NO: 29; and the VL CDR3 includes an amino acid sequence selected from the group consisting SEQ ID NO: 34.

An example VH sequence includes the amino acid sequence of SEQ ID NO: 3. An example VL sequence includes the amino acid sequence of SEQ ID NO: 4. In some embodiments, the VH includes the amino acid sequence of SEQ ID NO: 3, or a sequence having at least 75%, 80%, 85%, 90%, 95% or 99% sequence identity to SEQ ID NO: 3, while retaining the corresponding VH CDRs. In some embodiments, the VL includes an amino acid sequence of SEQ ID NO: 4, or a sequence having at least 75%, 80%, 85%, 90%, 95% or 99% sequence identity to SEQ ID NO: 4, while retaining the corresponding VL CDRs.

Also provided, in some embodiments, are antibodies and antigen-binding fragments therefore that bind to the same epitope on MerTK as 85H8D5. Also provided, in some embodiments, are antibodies and antigen-binding fragments therefore that competes with 85H8D5 in binding to MerTK.

In some embodiments, provided is an antibody or antigen-binding fragment that is derived from antibody 216G3D6. In some embodiments, the VH CDR1 includes the amino acid sequence of SEQ ID NO: 35; the VH CDR2 includes the amino acid sequence of SEQ ID NO: 36; the VH CDR3 includes the amino acid sequence of SEQ ID NO: 37; the VL CDR1 includes the amino acid sequence of SEQ ID NO: 38; the VL CDR2 includes the amino acid sequence of SEQ ID NO: 39; and the VL CDR3 includes an amino acid sequence selected from the group consisting SEQ ID NO: 40.

An example VH sequence includes the amino acid sequence of SEQ ID NO: 5. An example VL sequence includes the amino acid sequence of SEQ ID NO: 6. In some embodiments, the VH includes the amino acid sequence of SEQ ID NO: 5, or a sequence having at least 75%, 80%, 85%, 90%, 95% or 99% sequence identity to SEQ ID NO: 5, while retaining the corresponding VH CDRs. In some embodiments, the VL includes an amino acid sequence of SEQ ID NO: 6, or a sequence having at least 75%, 80%, 85%, 90%, 95% or 99% sequence identity to SEQ ID NO: 6, while retaining the corresponding VL CDRs.

Also provided, in some embodiments, are antibodies and antigen-binding fragments therefore that bind to the same epitope on MerTK as 216G3D6. Also provided, in some embodiments, are antibodies and antigen-binding fragments therefore that competes with 216G3D6 in binding to MerTK.

In some embodiments, provided is an antibody or antigen-binding fragment that is derived from antibody 247E5A8. In some embodiments, the VH CDR1 includes the amino acid sequence of SEQ ID NO: 41; the VH CDR2 includes the amino acid sequence of SEQ ID NO: 42; the VH CDR3 includes the amino acid sequence of SEQ ID NO: 43; the VL CDR1 includes the amino acid sequence of SEQ ID NO: 44; the VL CDR2 includes the amino acid sequence of SEQ ID NO: 45; and the VL CDR3 includes an amino acid sequence selected from the group consisting SEQ ID NO: 46.

An example VH sequence includes the amino acid sequence of SEQ ID NO: 7. An example VL sequence includes the amino acid sequence of SEQ ID NO: 8. In some embodiments, the VH includes the amino acid sequence of SEQ ID NO: 7, or a sequence having at least 75%, 80%, 85%, 90%, 95% or 99% sequence identity to SEQ ID NO: 7, while retaining the corresponding VH CDRs. In some embodiments, the VL includes an amino acid sequence of SEQ ID NO: 8, or a sequence having at least 75%, 80%, 85%, 90%, 95% or 99% sequence identity to SEQ ID NO: 8, while retaining the corresponding VL CDRs.

Also provided, in some embodiments, are antibodies and antigen-binding fragments therefore that bind to the same epitope on MerTK as 247E5A8. Also provided, in some embodiments, are antibodies and antigen-binding fragments therefore that competes with 247E5A8 in binding to MerTK.

In some embodiments, provided is an antibody or antigen-binding fragment that is derived from antibody 252C12B10. In some embodiments, the VH CDR1 includes the amino acid sequence of SEQ ID NO: 47; the VH CDR2 includes the amino acid sequence of SEQ ID NO: 48; the VH CDR3 includes the amino acid sequence of SEQ ID NO: 49; the VL CDR1 includes the amino acid sequence of SEQ ID NO: 50; the VL CDR2 includes the amino acid sequence of SEQ ID NO: 51; and the VL CDR3 includes an amino acid sequence selected from the group consisting SEQ ID NO: 52.

An example VH sequence includes the amino acid sequence of SEQ ID NO: 9. An example VL sequence includes the amino acid sequence of SEQ ID NO: 10. In some embodiments, the VH includes the amino acid sequence of SEQ ID NO: 9, or a sequence having at least 75%, 80%, 85%, 90%, 95% or 99% sequence identity to SEQ ID NO: 9, while retaining the corresponding VH CDRs. In some embodiments, the VL includes an amino acid sequence of SEQ ID NO: 10, or a sequence having at least 75%, 80%, 85%, 90%, 95% or 99% sequence identity to SEQ ID NO: 10, while retaining the corresponding VL CDRs.

Also provided, in some embodiments, are antibodies and antigen-binding fragments therefore that bind to the same epitope on MerTK as 252C12B10. Also provided, in some embodiments, are antibodies and antigen-binding fragments therefore that competes with 252C12B10 in binding to MerTK.

In some embodiments, provided is an antibody or antigen-binding fragment that is derived from antibody 252H9D5. In some embodiments, the VH CDR1 includes the amino acid sequence of SEQ ID NO: 53; the VH CDR2 includes the amino acid sequence of SEQ ID NO: 54; the VH CDR3 includes the amino acid sequence of SEQ ID NO: 55; the VL CDR1 includes the amino acid sequence of SEQ ID NO: 56; the VL CDR2 includes the amino acid sequence of SEQ ID NO: 57; and the VL CDR3 includes an amino acid sequence selected from the group consisting SEQ ID NO: 58.

An example VH sequence includes the amino acid sequence of SEQ ID NO: 11. An example VL sequence includes the amino acid sequence of SEQ ID NO: 12. In some embodiments, the VH includes the amino acid sequence of SEQ ID NO: 11, or a sequence having at least 75%, 80%, 85%, 90%, 95% or 99% sequence identity to SEQ ID NO: 11, while retaining the corresponding VH CDRs. In some embodiments, the VL includes an amino acid sequence of SEQ ID NO: 12, or a sequence having at least 75%, 80%, 85%, 90%, 95% or 99% sequence identity to SEQ ID NO: 12, while retaining the corresponding VL CDRs.

Also provided, in some embodiments, are antibodies and antigen-binding fragments therefore that bind to the same epitope on MerTK as 252H9D5. Also provided, in some embodiments, are antibodies and antigen-binding fragments therefore that competes with 252H9D5 in binding to MerTK.

In some embodiments, provided is an antibody or antigen-binding fragment that is derived from antibody 254B4D9. In some embodiments, the VH CDR1 includes the amino acid sequence of SEQ ID NO: 59; the VH CDR2 includes the amino acid sequence of SEQ ID NO: 60; the VH CDR3 includes the amino acid sequence of SEQ ID NO: 61; the VL CDR1 includes the amino acid sequence of SEQ ID NO: 62; the VL CDR2 includes the amino acid sequence of SEQ ID NO: 63; and the VL CDR3 includes an amino acid sequence selected from the group consisting SEQ ID NO: 64.

An example VH sequence includes an amino acid sequence selected from the group consisting of SEQ ID NO: 13, 102 and 104. An example VL sequence includes an amino acid sequence selected from the group consisting of SEQ ID NO: 14 and 106.

In some embodiments, the VH includes an amino acid sequence of any one of SEQ ID NO: 13, 102 and 104, or a sequence having at least 75%, 80%, 85%, 90%, 95% or 99% sequence identity to any one of SEQ ID NO: 13, 102 and 104, while retaining the corresponding VH CDRs. In some embodiments, the VL includes an amino acid sequence of any one of SEQ ID NO: 14 and 106, or a sequence having at least 75%, 80%, 85%, 90%, 95% or 99% sequence identity to any one of SEQ ID NO: 14 and 106, while retaining the corresponding VL CDRs.

In some embodiments, the VH includes the amino acid sequence of SEQ ID NO: 102 and the VL includes the amino acid sequence of SEQ ID NO: 106. In some embodiments, the VH includes the amino acid sequence of SEQ ID NO: 104 and the VL includes the amino acid sequence of SEQ ID NO: 106.

Also provided, in some embodiments, are antibodies and antigen-binding fragments therefore that bind to the same epitope on MerTK as 254B4D9. Also provided, in some embodiments, are antibodies and antigen-binding fragments therefore that competes with 254B4D9 in binding to MerTK.

In some embodiments, provided is an antibody or antigen-binding fragment that is derived from antibody 265F11B5. In some embodiments, the VH CDR1 includes the amino acid sequence of SEQ ID NO: 65; the VH CDR2 includes the amino acid sequence of SEQ ID NO: 66; the VH CDR3 includes the amino acid sequence of SEQ ID NO: 67; the VL CDR1 includes the amino acid sequence of SEQ ID NO: 68; the VL CDR2 includes the amino acid sequence of SEQ ID NO: 69; and the VL CDR3 includes an amino acid sequence selected from the group consisting SEQ ID NO: 70.

An example VH sequence includes an amino acid sequence selected from the group consisting of SEQ ID NO: 15, and 93-96. An example VL sequence includes an amino acid sequence selected from the group consisting of SEQ ID NO: 16 and 98-100.

In some embodiments, the VH includes an amino acid sequence of any one of SEQ ID NO: 15, and 93-96, or a sequence having at least 75%, 80%, 85%, 90%, 95% or 99% sequence identity to any one of SEQ ID NO: 15, and 93-96, while retaining the corresponding VH CDRs. In some embodiments, the VL includes an amino acid sequence of any one of SEQ ID NO: 16 and 98-100, or a sequence having at least 75%, 80%, 85%, 90%, 95% or 99% sequence identity to any one of SEQ ID NO: 16 and 98-100, while retaining the corresponding VL CDRs.

In some embodiments, the VH includes the amino acid sequence of SEQ ID NO: 95 and the VL includes an amino acid sequence of any one of SEQ ID NO: 16 and 98-100. In some embodiments, the VH includes the amino acid sequence of SEQ ID NO: 95 and the VL includes the amino acid sequence of SEQ ID NO: 99.

In some embodiments, the VH includes the amino acid sequence of SEQ ID NO: 96 and the VL includes an amino acid sequence of any one of SEQ ID NO: 16 and 98-100. In some embodiments, the VH includes the amino acid sequence of SEQ ID NO: 96 and the VL includes the amino acid sequence of SEQ ID NO: 99.

In some embodiments, the VH includes an amino acid sequence of any one of SEQ ID NO: 15, and 93-96 and the VL includes the amino acid sequence of SEQ ID NO: 99. In some embodiments, the VH includes an amino acid sequence of any one of SEQ ID NO: 93-96 and the VL includes the amino acid sequence of SEQ ID NO: 99.

Also provided, in some embodiments, are antibodies and antigen-binding fragments therefore that bind to the same epitope on MerTK as 265F11B5. Also provided, in some embodiments, are antibodies and antigen-binding fragments therefore that competes with 265F11B5 in binding to MerTK.

In some embodiments, provided is an antibody or antigen-binding fragment that is derived from antibody 276C2D1. In some embodiments, the VH CDR1 includes the amino acid sequence of SEQ ID NO: 71; the VH CDR2 includes the amino acid sequence of SEQ ID NO: 66; the VH CDR3 includes the amino acid sequence of SEQ ID NO: 72; the VL CDR1 includes the amino acid sequence of SEQ ID NO: 73; the VL CDR2 includes the amino acid sequence of SEQ ID NO: 69; and the VL CDR3 includes an amino acid sequence selected from the group consisting SEQ ID NO: 74.

An example VH sequence includes the amino acid sequence of SEQ ID NO: 17. An example VL sequence includes the amino acid sequence of SEQ ID NO: 18. In some embodiments, the VH includes the amino acid sequence of SEQ ID NO: 17, or a sequence having at least 75%, 80%, 85%, 90%, 95% or 99% sequence identity to SEQ ID NO: 17, while retaining the corresponding VH CDRs. In some embodiments, the VL includes an amino acid sequence of SEQ ID NO: 18, or a sequence having at least 75%, 80%, 85%, 90%, 95% or 99% sequence identity to SEQ ID NO: 18, while retaining the corresponding VL CDRs.

Also provided, in some embodiments, are antibodies and antigen-binding fragments therefore that bind to the same epitope on MerTK as 276C2D1. Also provided, in some embodiments, are antibodies and antigen-binding fragments therefore that competes with 276C2D1 in binding to MerTK.

In some embodiments, provided is an antibody or antigen-binding fragment that is derived from antibody 280C6A3. In some embodiments, the VH CDR1 includes the amino acid sequence of SEQ ID NO: 65; the VH CDR2 includes the amino acid sequence of SEQ ID NO: 75; the VH CDR3 includes the amino acid sequence of SEQ ID NO: 67; the VL CDR1 includes the amino acid sequence of SEQ ID NO: 76; the VL CDR2 includes the amino acid sequence of SEQ ID NO: 69; and the VL CDR3 includes an amino acid sequence selected from the group consisting SEQ ID NO: 77.

An example VH sequence includes the amino acid sequence of SEQ ID NO: 19. An example VL sequence includes the amino acid sequence of SEQ ID NO: 20. In some embodiments, the VH includes the amino acid sequence of SEQ ID NO: 19, or a sequence having at least 75%, 80%, 85%, 90%, 95% or 99% sequence identity to SEQ ID NO: 19, while retaining the corresponding VH CDRs. In some embodiments, the VL includes an amino acid sequence of SEQ ID NO: 20, or a sequence having at least 75%, 80%, 85%, 90%, 95% or 99% sequence identity to SEQ ID NO: 20, while retaining the corresponding VL CDRs.

Also provided, in some embodiments, are antibodies and antigen-binding fragments therefore that bind to the same epitope on MerTK as 280C6A3. Also provided, in some embodiments, are antibodies and antigen-binding fragments therefore that competes with 280C6A3 in binding to MerTK.

In some embodiments, provided is an antibody or antigen-binding fragment that is derived from antibody 293C2B7. In some embodiments, the VH CDR1 includes the amino acid sequence of SEQ ID NO: 71; the VH CDR2 includes the amino acid sequence of SEQ ID NO: 78; the VH CDR3 includes the amino acid sequence of SEQ ID NO: 79; the VL CDR1 includes the amino acid sequence of SEQ ID NO: 73; the VL CDR2 includes the amino acid sequence of SEQ ID NO: 69; and the VL CDR3 includes an amino acid sequence selected from the group consisting SEQ ID NO: 74.

An example VH sequence includes the amino acid sequence of SEQ ID NO: 21. An example VL sequence includes the amino acid sequence of SEQ ID NO: 22. In some embodiments, the VH includes the amino acid sequence of SEQ ID NO: 21, or a sequence having at least 75%, 80%, 85%, 90%, 95% or 99% sequence identity to SEQ ID NO: 21, while retaining the corresponding VH CDRs. In some embodiments, the VL includes an amino acid sequence of SEQ ID NO: 22, or a sequence having at least 75%, 80%, 85%, 90%, 95% or 99% sequence identity to SEQ ID NO: 22, while retaining the corresponding VL CDRs.

Also provided, in some embodiments, are antibodies and antigen-binding fragments therefore that bind to the same epitope on MerTK as 293C2B7. Also provided, in some embodiments, are antibodies and antigen-binding fragments therefore that competes with 293C2B7 in binding to MerTK.

In some embodiments, provided is an antibody or antigen-binding fragment that is derived from antibody 300A5A3. In some embodiments, the VH CDR1 includes the amino acid sequence of SEQ ID NO: 65; the VH CDR2 includes the amino acid sequence of SEQ ID NO: 66; the VH CDR3 includes the amino acid sequence of SEQ ID NO: 80; the VL CDR1 includes the amino acid sequence of SEQ ID NO: 81; the VL CDR2 includes the amino acid sequence of SEQ ID NO: 69; and the VL CDR3 includes an amino acid sequence selected from the group consisting SEQ ID NO: 70.

An example VH sequence includes the amino acid sequence of SEQ ID NO: 23. An example VL sequence includes the amino acid sequence of SEQ ID NO: 24. In some embodiments, the VH includes the amino acid sequence of SEQ ID NO: 23, or a sequence having at least 75%, 80%, 85%, 90%, 95% or 99% sequence identity to SEQ ID NO: 23, while retaining the corresponding VH CDRs. In some embodiments, the VL includes an amino acid sequence of SEQ ID NO: 24, or a sequence having at least 75%, 80%, 85%, 90%, 95% or 99% sequence identity to SEQ ID NO: 24, while retaining the corresponding VL CDRs.

Also provided, in some embodiments, are antibodies and antigen-binding fragments therefore that bind to the same epitope on MerTK as 300A5A3. Also provided, in some embodiments, are antibodies and antigen-binding fragments therefore that competes with 300A5A3 in binding to MerTK.

Also provided, in some embodiments, are antibodies and antigen-binding fragments that include CDR sequences derived from the presently disclosed CDR sequences, with one, two or three amino acid substitutions, deletions, and/or additions.

In some embodiments, the antibody or fragment thereof is capable of inducing antibody-dependent cellular cytotoxicity (ADCC). In some embodiments, the antibody or fragment thereof is not capable of inducing antibody-dependent cellular cytotoxicity (ADCC).

### Multi-Functional Molecules

Multi-functional molecules that include an antibody or antigen-binding fragment specific to MerTK, such as those disclosed herein, and one or more antibody or antigen-binding fragment having specificity to a second antigen.

In some embodiments, the second antigen is a protein expressed on an immune cell, such as a T cell, a B cell, a monocyte, a macrophage, a neutrophil, a dendritic cell, a phagocyte, a natural killer cell, an eosinophil, a basophil, and a mast cell.

In some embodiments, the second antigen is CD3, CD47, PD1, PD-L1, LAG3, TIM3, CTLA4, VISTA, CSFR1, A2AR, CD73, CD39, CD40, CEA, HER2, CMET, 4-1BB, OX40, SIRPA CD16, CD28, ICOS, CTLA4, BTLA, TIGIT, HVEM, CD27, VEGFR, or VEGF. In one embodiment, the second antigen is PD1. In one embodiment, the second antigen is PD-L1.

Different formats of bispecific antibodies are also provided. In some embodiments, each of the anti-MerTK fragment and the second fragment each is independently selected from a Fab fragment, a single-chain variable fragment (scFv), or a single-domain antibody. In some embodiments, the bispecific antibody further includes a Fc fragment.

Bifunctional molecules that include not just antibody or antigen binding fragment are also provided. As a tumor antigen targeting molecule, an antibody or antigen-binding fragment specific to MerTK, such as those described here, can be combined with an immune cytokine or ligand optionally through a peptide linker. The linked immune cytokines or ligands include, but not limited to, IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL. Such bi-functional molecules can combine the immune checkpoint blocking effect with tumor site local immune modulation.

### Chimeric Antigen Receptors

Also provided, in one embodiment, is a chimeric antigen receptor (CAR) that includes the antibody or fragment thereof of the present disclosure as a targeting unit. In some embodiments, the CAR includes an antibody or fragment thereof of the present disclosure, a transmembrane domain, a costimulatory domain, and a CD34 intracellular domain.

A transmembrane domain can be designed to be fused to the extracellular domain which includes the antibody or fragment, optionally through a hinge domain. It can similarly be fused to an intracellular domain, such as a costimulatory domain. In some embodiments, the transmembrane domain can include the natural transmembrane region of a costimulatory domain (e.g., the TM region of a CD28T or 4-1BB employed as a costimulatory domain) or the natural transmembrane domain of a hinge region (e.g., the TM region of a CD8 alpha or CD28T employed as a hinge domain).

In some embodiments, the transmembrane domain can include a sequence that spans a cell membrane, but extends into the cytoplasm of a cell and/or into the extracellular space. For example, a transmembrane can include a membrane-spanning sequence which itself can further include 1, 2, 3, 4, 5, 6, 7, 8, 9, 10 or more amino acids that extend into the cytoplasm of a cell, and/or the extracellular space. Thus, a transmembrane domain includes a membrane-spanning region, yet can further comprise an amino acid(s) that extend beyond the internal or external surface of the membrane itself; such sequences can still be considered to be a “transmembrane domain”.

In some embodiments, the transmembrane domain is fused to the cytoplasmic domain through a short linker. Optionally, the short peptide or polypeptide linker, preferably between 2 and 10 amino acids in length can form the linkage between the transmembrane domain and a proximal cytoplasmic signaling domain of the chimeric receptor. A glycine-serine doublet (GS), glycine-serine-glycine triplet (GSG), or alanine-alanine-alanine triplet (AAA) provides a suitable linker.

In some embodiments, the CAR further includes a costimulatory domain. In some embodiments, the costimulatory domain is positioned between the transmembrane domain and an activating domain. Example costimulatory domains include, but are not limited to, CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD18 (ITGB2), CD19 (B4), CD27 (T FRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD 100 (SEMA4D), CD 103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KTR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (TNFRSF 18), inducible T cell co-stimulator (ICOS), LFA-1 (CD 1 la/CD 18), KG2C, DAP-10, ICAM-1, Kp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

In some embodiments, the cytoplasmic portion of the CAR also includes a signaling/activation domain. In one embodiment, the signaling/activation domain is the CD3ξ domain, or is an amino acid sequence having at least about 80%, 85%, 90%, 95%, 98% or 99% sequence identity to the CD3ξ domain.

Polynucleotides, mRNA, and Methods of Expressing or Preparing Antibodies

The present disclosure also provides polynucleotides or nucleic acid molecules encoding the antibodies, variants or derivatives thereof of the disclosure, or the CAR. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

In some embodiments, the polynucleotide is an mRNA molecule. In some embodiments, the mRNA can be introduced into a target cell for expressing the antibody or fragment thereof.

mRNAs may be synthesized according to any of a variety of known methods. For example, the mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, for the preparation of antibody-coding mRNA, a DNA template is transcribed in vitro. A suitable DNA template typically has a promoter, for example a T3, T7 or SP6 promoter, for in vitro transcription, followed by desired nucleotide sequence for desired antibody encoding (e.g., heavy chain or light chain encoding) mRNA and a termination signal.

Desired antibody encoding (e.g., heavy chain or light chain encoding) mRNA sequence may be determined and incorporated into a DNA template using standard methods. For example, starting from a desired amino acid sequence (e.g., a desired heavy chain or light chain sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

The mRNA may be synthesized as unmodified or modified mRNA. Typically, mRNAs are modified to enhance stability. Modifications of mRNA can include, for example, modifications of the nucleotides of the RNA. A modified mRNA can thus include, for example, backbone modifications, sugar modifications or base modifications. In some embodiments, antibody encoding mRNAs (e.g., heavy chain and light chain encoding mRNAs) may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 13-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosure of which is included here in its full scope by reference.

In some embodiments, the mRNAs (e.g., heavy chain and light chain encoding mRNAs) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, the mRNAs (e.g., heavy chain and light chain encoding mRNAs) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, the mRNAs (e.g., heavy chain and light chain encoding mRNAs) may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, the mRNAs (e.g., heavy chain and light chain encoding mRNAs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5'S triphosphate linkage; and the 7-nitrogen of guanine is then methylated by a methyltransferase. Examples of cap structures include, but are not limited to, m7G(5′)ppp (5′(A,G(5′)ppp(5)A and G(5)ppp(5′)G.

In some embodiments, the mRNAs (e.g., heavy chain and light chain encoding mRNAs) include a 3′ poly(A) tail structure. A poly-A tail on the 3′ terminus of mRNA typically includes about 10 to 300 adenosine nucleotides (e.g., about 10 to 200 adenosine nucleotides, about 10 to 175 adenosine nucleotides, about 10 to 150 adenosine nucleotides, about 10 to 125 adenosine nucleotides, 10 to 100 adenosine nucleotides, about 10 to 75 adenosine nucleotides, about 20 to 70 adenosine nucleotides, or about 20 to 60 adenosine nucleotides). In some embodiments, antibody encoding mRNAs (e.g., heavy chain and light chain encoding mRNAs) include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the mRNAs (e.g., heavy chain and light chain encoding mRNAs) include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length (e.g., about 50 and 400 nucleotides in length, about 50 and 300 nucleotides in length, about 50 and 200 nucleotides in length, or about 50 and 100 nucleotides in length).

In some embodiments, a 5′ region of an mRNA (e.g., heavy chain and light chain encoding mRNAs) includes a sequence encoding a signal peptide, such as those described herein. In particular embodiments, a signal peptide derived from human growth hormone (hGH) is incorporated in the 5′ region. Typically, a signal peptide encoding sequence is linked, directly or indirectly, to the heavy chain or light chain encoding sequence at the N-terminus.

The present technology may be used to deliver any antibody known in the art and antibodies that can be produced against desired antigens using standard methods. The present invention may be used to deliver monoclonal antibodies, polyclonal antibodies, antibody mixtures or cocktails, human or humanized antibodies, chimeric antibodies, or bi-specific antibodies.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment and Uses

As described herein, the antibodies, variants, or derivatives of the present disclosure may be used in certain treatment and diagnostic methods.

The present disclosure is further directed to antibody-based therapies which involve administering the antibodies or fragments of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. As provided above, MerTK is rarely expressed in normal adult tissues, but is present at high levels in placenta and in most common tumors, typically more than 80% of carcinomas of the kidney, breast, colon, prostate, and ovary.

Accordingly, in some embodiments, provided are methods for treating a cancer in a patient in need thereof. The method, in one embodiment, entails administering to the patient an effective amount of an antibody or fragment of the present disclosure. In some embodiments, at least one of the cancer cells (e.g., stromal cells) in the patient over-express MerTK.

Cellular therapies, such as chimeric antigen receptor (CAR) T-cell therapies, are also provided in the present disclosure. A suitable cell can be used, that is transduced with a vector that encodes, or put in contact with, a CAR that includes an anti-MerTK antibody of the present disclosure (or alternatively engineered to express an anti-MerTK antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, the antibody, polynucleotide, or composition of the present disclosure can be used for treating inflammatory disease or conditions. In some embodiments, the inflammatory disease or condition to be treated by the disclosed antibodies, fragments and compositions includes one or more of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, and ulcerative colitis.

In some embodiments, the autoimmune disease or condition to be treated by the disclosed antibodies, fragments and compositions includes one or more of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barrésyndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. While the cause of rheumatoid arthritis is not clear, it is believed to involve a combination of genetic and environmental factors. The underlying mechanism involves the body's immune system attacking the joints. This results in inflammation and thickening of the joint capsule. The goals of treatment are to reduce pain, decrease inflammation, and improve a person's overall functioning. Pain medications, steroids, and NSAIDs are frequently used to help with symptoms. A group of medications called disease-modifying antirheumatic drugs (DMARDs), such as hydroxychloroquine and methotrexate, may be used to try to slow the progression of disease.

Osteoarthritis (OA) is a type of joint disease that results from breakdown of joint cartilage and underlying bone. The most common symptoms are joint pain and stiffness. Initially, symptoms may occur only following exercise, but over time may become constant. Other symptoms may include joint swelling, decreased range of motion, and when the back is affected weakness or numbness of the arms and legs. Causes include previous joint injury, abnormal joint or limb development, and inherited factors. Risk is greater in those who are overweight, have one leg of a different length, and have jobs that result in high levels of joint stress. Osteoarthritis is believed to be caused by mechanical stress on the joint and low grade inflammatory processes. Treatment includes exercise, efforts to decrease joint stress, support groups, and pain medications.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. While the cause is not clear, the underlying mechanism is thought to be either destruction by the immune system or failure of the myelin-producing cells. There is no known cure for multiple sclerosis. Treatments attempt to improve function after an attack and prevent new attacks.

Asthma is a common long-term inflammatory disease of the airways of the lungs. It is characterized by variable and recurring symptoms, reversible airflow obstruction, and bronchospasm. Symptoms include episodes of wheezing, coughing, chest tightness, and shortness of breath. Asthma is thought to be caused by a combination of genetic and environmental factors. Environmental factors include exposure to air pollution and allergens. Asthma is classified according to the frequency of symptoms, forced expiratory volume in one second (FEVI), and peak expiratory flow rate. It may also be classified as atopic or non-atopic, where atopy refers to a predisposition toward developing a type 1 hypersensitivity reaction. There is no cure for asthma. Symptoms can be prevented by avoiding triggers, such as allergens and irritants, and by the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases, intravenous corticosteroids, magnesium sulfate, and hospitalization may be required.

Chronic obstructive pulmonary disease (COPD) is a type of obstructive lung disease characterized by long-term poor airflow. COPD can include two main conditions, emphysema and chronic bronchitis. In emphysema, the walls between many of the air sacs are damaged. As a result, the air sacs lose their shape and become floppy. This damage also can destroy the walls of the air sacs, leading to fewer and larger air sacs instead of many tiny ones. If this happens, the amount of gas exchange in the lungs is reduced. In chronic bronchitis, the lining of the airways stays constantly irritated and inflamed, and this causes the lining to swell. Lots of thick mucus forms in the airways, making it hard to breathe. There is no known cure for COPD, but the symptoms are treatable and its progression can be delayed.

Pain is a distressing feeling often caused by intense or damaging stimuli, such as stubbing a toe, burning a finger, putting alcohol on a cut, or bumping the “funny bone”. Pain is a complex, subjective phenomenon, defining pain has been a challenge. Pain is also referred to as an unpleasant sensory and emotional experience associated with actual or potential tissue damage. Pain is sometimes regarded as a symptom of an underlying condition, such as inflammation.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibody or fragment include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracisternally, intravaginally, intraperitoneally, topically (as by powders, ointments, drops or transdermal patch), buccally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies or fragments of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antibodies or fragments of the present disclosure is typically 0.001 mg/kg to 100 mg/kg of the patient's body weight, between 0.01 mg/kg and 20 mg/kg of the patient's body weight, or 0.5 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody or fragment and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Generation of Mouse Monoclonal Antibodies (mAb) Against Human MerTK

This example describes preparation of anti-human MerTK mouse monoclonal antibodies using the hybridoma technology.

Antigen: human MerTK-Fc protein and human MerTK-his protein comprising the extracellular domain (ECD) of human MerTK fused to human IgG1 Fe or his tag at the C-terminal.

Immunization: To generate mouse monoclonal antibodies to human MerTK, Balb/c mice, SJL mice, C57BL/6 mice and SD mice were immunized with human MerTK-Fc or MerTK-his protein at biweekly intervals intraperitoneally and subcutaneously. Serum titers of immunized mice were monitored by ELISA against human MerTK-his and cyno MerTK-his protein and FACS against human MerTK overexpressed on CHO-K1 cell line (CHO-K1-hMerTK) while CHO-K1 parental cell line served as the negative control. After 2-4 rounds of immunization, mice with sufficient titers were boosted with 25 g human MerTK-his protein and selected for fusions.

Cell fusion and Hybridoma screening: Splenocytes from the selected mice were fused with mouse myeloma cell line Sp2/0 by electrofusion. The supernatants of the hybridoma cells were first screened by ELISA against human MerTK-his protein. Then, the supernatants of positive clones were screened by ELISA against cyno MerTK-his and for function in blocking MerTK binding to its ligand growth arrest-specific protein 6 (Gas6) in an ELISA-based receptor blocking assay. Positive primary clones from each fusion were subcloned, and subclones were further confirmed by ELISA-based receptor blocking assay and affinity ranking. Hybridoma clones with efficient binding and blocking abilities were sequenced and selected for further analysis. The amino acid sequences of the variable regions of these clones are provided in Table 1 below.

TABLE 1
Sequences of the variable regions of selected clones (underlining/bold indicates CDR)

| Name | Sequence | SEQ ID NO: |
|---|---|---|
| 10F7D9 VH | QIQLVQSGPELKKPGETVKISCKASGYTFTNYGMNWVKQAPGKGLK | 1 |
| | WMGWINTYTGEPTYADDFKGRFAFSLETSASTAYLQINNLKNEDLA | |
| | TYFCATYGHYAWFAYWGQGTLVTVSA | |
| 10F7D9 VL | QIVLSQSPAILSASPGEKVTMTCRASSSVSYMHWYQQKSGSSPKPW | 2 |
| | IYATSNLASGVPARFSGSGSGTSYSLTISRVEAEDAATYFCQQWGT | |
| | KPLTFGAGTKLELK | |
| 85H8D5 VH | QIQLVQSGPELKKPGETVKISCMASGYTFTTHGVTWVKQAPGQGLQ | 3 |
| | RMGWINTYSGVPTYADDFKGRFVFSLETSANTAHLQINNLKNEDTA | |
| | TYFCARTNNYRYFDVWGTGTTVTVSS | |
| 85H8D5 VL | QIVLSQSPAILSSSPGEKVTMTCRASSSVSYMHWYQQKPGSSPKPW | 4 |
| | IYATSNLASGVPARFSGSGSGTSYFLTISRVEAEDAATYYCQQWNT | |
| | DPPTFGGGTKLEIK | |
| 216G3D6 VH | QVTLKESGPGILQPSQTLSLTCSFSGFSLSTSGMGVGWIRQPSGKG | 5 |
| | LEWLAHIWWDDDKHCNPALKSRLTISKDTSSNQVFLKIASLDTADT | |
| | ATYYCTQMTMTREFAYWGQGTLVTVSA | |
| 216G3D6 VL | DIQMTQTISSLSASLGDRVTISCSAGQGISNYLNWYQQKPDGTVKL | 6 |
| | LIYYTSRLHSGVPSRFSGSGSGTDFSLTISNLEPEDIATYFCQQYS | |
| | KFPYTFGGGTKLEIK | |
| 247E5A8 VH | DVQLQESGPGLVKPSQSLSLTCSVTGYSITGGYYWTWIRQFPGNKL | 7 |
| | EWMGYISYDGDNNYNPSLKNRISITRDTSKNQFFLNLNSVTTEDTA | |
| | TYYCAREGGRLRTMDYWGQGTSVTVSS | |
| 247E5A8 VL | QIVLTQSPAIMSASPGEKVTITCRASSSVNYMHWFQQKPGTSPKLW | 8 |
| | IYTTSNLASGVPARFSGSGSGTSYSLTISRMEAEDAATYYCQQRSS | |
| | YPFTFGSGTKLEIK | |
| 252C12B10 VH | QVQLQQPGTELVKPGASVKLSCKTSGYTFTSYWMHWMKQRPGQGLE | 9 |
| | WLGHINPSTGGSNYNEKFKSKATLTVDKSSNTAYMQLSSLTSEDSA | |
| | VYYCARPRGFYGYSYWYFDVWGSGTTVTVSS | |
| 252C12B10 VL | DIQMTQTTSSLSASLGDRVTISCSASQDINNYLNWYQHKPDGTIKV | 10 |
| | LIYFTSSLHSGVPSRFSGSGSGTEYSLTISNLEPEDIATYYCQQYS | |
| | KLPPTFGSGTKLEIK | |
| 252H9D5 VH | QVTLRESGPGILQPSQTLSLTCSFSGFSLNTFGMGVGWIRQSSGKG | 11 |
| | LKWLAHIWWDDDKFYNPALKSRLTISKDTSKNQVFLKIANVDTTDT | |
| | ATYYCARIEGGLPYYYDYWGQGTPLIVSS | |
| 252H9D5 VL | DIVMTQSQKIMSTSVGDRISVTCKASQIVGTNVVWYQQKPGQSPKA | 12 |
| | LIYSTSYRYSGVPDRFTGSRSGTDFTLTISNVQSEDLANYFCQQYN | |
| | SYPFTFGSGTKLEIK | |
| 254B4D9 VH | QIQLVQSGPELKKPGETVKISCKASGYTFTAYGMSWVKQAPGKGLQ | 13 |
| | WMGWINTYSGVPTYTDDFKGRFAFSLEASASTAFLQINNLKNEDTS | |
| | TYFCARRAAVVAKYWYFDVWGTGTTVTVSS | |
| 254B4D9 VL | DIQMTQSPSSLFASLGGKVIITCKSSQDINKRISWYQHKPGKGPRL | 14 |
| | LIHSTSSLQPGIPLRFSGSGSGRDYSFSISNLEPEDIATYFCLQYD | |
| | NLLTFGAGTKLELK | |
| 265F11B5 VH | QVQLQQSGPELVKPGASVRLSCKASGYTFTTYDINWVKQRPGLGLE | 15 |
| | WIGWIYPRDGYTKYNEKFKGRATLAVDTSSSTAYMELHSLTSEDSA | |
| | VYFCTRGYYDNLYYFDYWGQGTTLTVSS | |
| 265F11B5 VL | EIVLTQSPTTVAASPGEKITITCSASSIISSNYFHWYQQRPGFSPK | 16 |
| | LLIYRTSNLASGVPARFSGSGSGTSYSLTIGTMEAEDVATYYCQQG | |
| | RSVPLTFGAGTKLELK | |
| 276C2D1 VH | QVQLQQSGPELVKPGASVKLSCKASGYTFTSYDINWVKQRPGLGLE | 17 |
| | WIGWIYPRDGYTKYNEKFKGKATLAVDTSSSTAYMELHSLTSEDSA | |
| | VYFCARGYYSNLYYFDYWGQGTTLTVSS | |
| 276C2D1 VL | EVVLTQSPPTMAASPGEKITITCSASSSISSNYLHWYQQKPGFSPK | 18 |
| | LLIYRTSNLASGVPARFSGSGSGTSYSLTIGTMEAEDVATYYCQQG | |
| | RSIPLTFGAGTKLELK | |
| 280C6A3 VH | QVQLQQSGPELVKPGASVKLSCKASGYSFTTYDINWVKQRPGQGLE | 19 |
| | WIGWIYPRDGSTKYNEKFKGKATLTLDTSSNTAYMGLHSLTSEDSA | |
| | VYFCARGYYDNLYYFDYWGQGTTLTVSS | |
| 280C6A3 VL | EIVLTQSPTTMAASPGEKITITCSASSSVSSNYLHWYQQKPGFSPK | 20 |
| | LLIYRTSNLASGVPARFSGSGSGTSYSLTIGTMEAEDVATYYCQQG | |
| | RIVPLTFGAGTKLELK | |
| 293C2B7 VH | QVQLQQSGPELVKPGASVKLSCKASGYTFTSYDINWVKQRPGQGLE | 21 |
| | WIGWIYPRDGYTKYKEKFKGKATLTVDTSSSTAYMELHSLTSEDSA | |
| | VYFCARGFYSNLYYFDYWGQGTTLTVSS | |
| 293C2B7 VL | EIVLTQSPTTMATSPGEKITITCSASSSISSNYLHWYQQKPGFSPK | 22 |
| | LLIYRTSNLASGVPDRFSGSGSGTSYSLTIGTMEAEDVATYYCQQG | |
| | RSIPLTFGAGTKLELK | |
| 300A5A3 VH | QVQLQQSGPELVKPGASVKLSCKASGYTFTTYDINWVKQRPGLGLE | 23 |
| | WIGWIYPRDGYTKYNEKFKGKATLAVDTSSSTASMELHSLTSEDSA | |
| | VYFCARGYYDNLYYFDNWGPGTTLTVSS | |
| 300A5A3 VL | ENVLTQSPTTVAASPGEKITITCSASSIISSNYLHWYQQKPGFSPK | 24 |
| | LLIYRTSNLASGVPARFSGSGSGTSYSLTIGTMEAEDAATYYCQQG | |
| | RSVPLTFGAGTKLELK | |

TABLE 1A
CDR sequences of selected clones

| CDRs | Sequence | SEQ ID NO: |
|---|---|---|
| 10F7D9 VH CDR1 | NYGMN | 25 |
| 10F7D9 VH CDR2 | WINTYTGEPTYADDFKG | 26 |
| 10F7D9 VH CDR3 | YGHYAWFAY | 27 |
| 10F7D9 VL CDR1 | RASSSVSYMH | 28 |
| 10F7D9 VL CDR2 | ATSNLAS | 29 |
| 10F7D9 VL CDR3 | QQWGTKPLT | 30 |
| 85H8D5 VH CDR1 | THGVT | 31 |
| 85H8D5 VH CDR2 | WINTYSGVPTYADDFKG | 32 |
| 85H8D5 VH CDR3 | TNNYRYFDV | 33 |
| 85H8D5 VL CDR1 | RASSSVSYMH | 28 |
| 85H8D5 VL CDR2 | ATSNLAS | 29 |
| 85H8D5 VL CDR3 | QQWNTDPPT | 34 |
| 216G3D6 VH CDR1 | TSGMGVG | 35 |
| 216G3D6 VH CDR2 | HIWWDDDKHCNPALKS | 36 |
| 216G3D6 VH CDR3 | MTMTREFAY | 37 |
| 216G3D6 VL CDR1 | SAGQGISNYLN | 38 |
| 216G3D6 VL CDR2 | YTSRLHS | 39 |
| 216G3D6 VL CDR3 | QQYSKFPYT | 40 |
| 247E5A8 VH CDR1 | GGYYWT | 41 |
| 247E5A8 VH CDR2 | YISYDGDNNYNPSLKN | 42 |
| 247E5A8 VH CDR3 | EGGRLRTMDY | 43 |
| 247E5A8 VL CDR1 | RASSSVNYMH | 44 |
| 247E5A8 VL CDR2 | TTSNLAS | 45 |
| 247E5A8 VL CDR3 | QQRSSYPFT | 46 |
| 252C12B10 VH CDR1 | SYWMH | 47 |
| 252C12B10 VH CDR2 | HINPSTGGSNYNEKFKS | 48 |
| 252C12B10 VH CDR3 | PRGFYGYSYWYFDV | 49 |
| 252C12B10 VL CDR1 | SASQDINNYLN | 50 |
| 252C12B10 VL CDR2 | FTSSLHS | 51 |
| 252C12B10 VLCDR3 | QQYSKLPPT | 52 |
| 252H9D5 VH CDR1 | TFGMGVG | 53 |
| 252H9D5 VH CDR2 | HIWWDDDKFYNPALKS | 54 |
| 252H9D5 VH CDR3 | IEGGLPYYYDY | 55 |
| 252H9D5 VL CDR1 | KASQIVGTNVV | 56 |
| 252H9D5 VL CDR2 | STSYRYS | 57 |
| 252H9D5 VL CDR3 | QQYNSYPFT | 58 |
| 254B4D9 VH CDR1 | AYGMS | 59 |
| 254B4D9 VH CDR2 | WINTYSGVPTYTDDFKG | 60 |
| 254B4D9 VH CDR3 | RAAVVAKYWYFDV | 61 |
| 254B4D9 VL CDR1 | KSSQDINKRIS | 62 |
| 254B4D9 VL CDR2 | STSSLQP | 63 |
| 254B4D9 VL CDR3 | LQYDNLLT | 64 |
| 265F11B5 VH CDR1 | TYDIN | 65 |
| 265F11B5 VH CDR2 | WIYPRDGYTKYNEKFKG | 66 |
| 265F11B5 VH CDR3 | GYYDNLYYFDY | 67 |
| 265F11B5 VL CDR1 | SASSIISSNYFH | 68 |
| 265F11B5 VL CDR2 | RTSNLAS | 69 |
| 265F11B5 VL CDR3 | QQGRSVPLT | 70 |
| 276C2D1 VH CDR1 | SYDIN | 71 |
| 276C2D1 VH CDR2 | WIYPRDGYTKYNEKFKG | 66 |
| 276C2D1 VH CDR3 | GYYSNLYYFDY | 72 |
| 276C2D1 VL CDR1 | SASSSISSNYLH | 73 |
| 276C2D1 VL CDR2 | RTSNLAS | 69 |
| 276C2D1 VL CDR3 | QQGRSIPLT | 74 |
| 280C6A3 VH CDR1 | TYDIN | 65 |
| 280C6A3 VH CDR2 | WIYPRDGSTKYNEKFKG | 75 |
| 280C6A3 VH CDR3 | GYYDNLYYFDY | 67 |
| 280C6A3 VL CDR1 | SASSSVSSNYLH | 76 |
| 280C6A3 VL CDR2 | RTSNLAS | 69 |
| 280C6A3 VL CDR3 | QQGRIVPLT | 77 |
| 293C2B7 VH CDR1 | SYDIN | 71 |
| 293C2B7 VH CDR2 | WIYPRDGYTKYKEKFKG | 78 |
| 293C2B7 VH CDR3 | GFYSNLYYFDY | 79 |
| 293C2B7 VL CDR1 | SASSSISSNYLH | 73 |
| 293C2B7 VL CDR2 | RTSNLAS | 69 |
| 293C2B7 VL CDR3 | QQGRSIPLT | 74 |
| 300A5A3 VH CDR1 | TYDIN | 65 |
| 300A5A3 VH CDR2 | WIYPRDGYTKYNEKFKG | 66 |
| 300A5A3 VH CDR3 | GYYDNLYYFDN | 80 |
| 300A5A3 VL CDR1 | SASSIISSNYLH | 81 |
| 300A5A3 VL CDR2 | RTSNLAS | 69 |
| 300A5A3 VL CDR3 | QQGRSVPLT | 70 |

### Example 2: Antigen Binding Properties of Anti-MerTK Chimeric Monoclonal Antibodies

The variable regions of the mouse MerTK antibody were fused to the human IgG1 constant region containing L234A/L235A/P329G (LALAPG) mutations to generate chimeric monoclonal antibodies with abolished Fc binding abilities. Benchmark antibodies including M6, Ab2000-A7 and h13B4.v16 were generated individually as above using sequences described in patent applications WO2019084307A1/WO2016106221A1/WO2020214995A1. This example tested the binding properties of the anti-MerTK chimeric monoclonal antibodies.

### ELISA Binding Activities of Anti-MerTK Chimeric Antibodies to Human and Cyno MerTK Protein

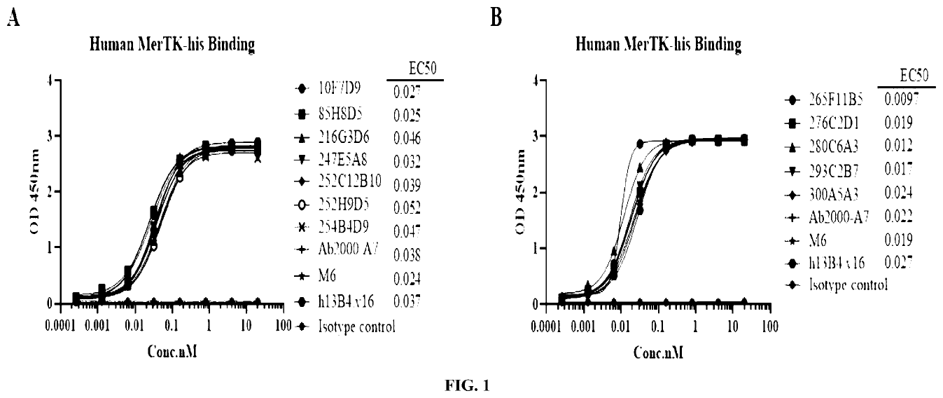

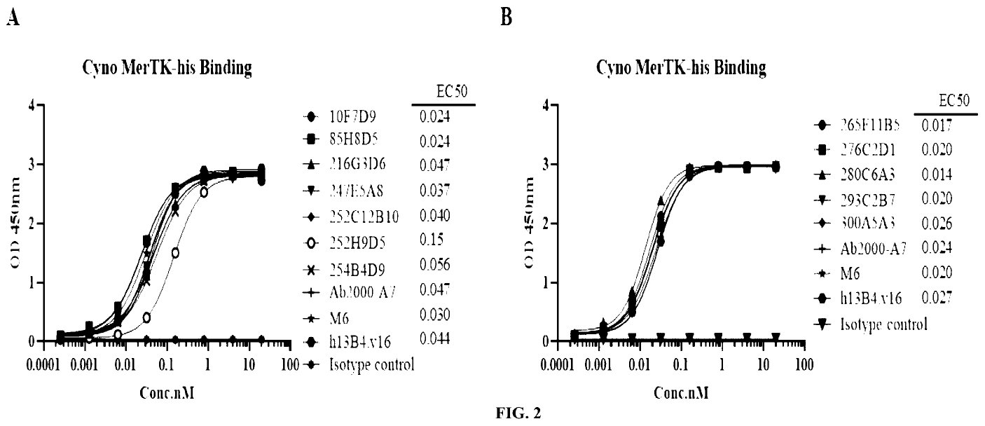

To evaluate the binding activity, the chimeric mAbs were subjected to ELISA test. Briefly, 96-well plates were coated with human or cyno MerTK-his protein at 2 μg/mL in PBS, 100 μL/well at 4° C. overnight, then blocked with 150 μL/well of 1% BSA. Five-fold dilutions of MerTK antibodies starting from 20 nM were added to each well and incubated for 1 hour at room temperature. The plates were washed with PBS/Tween-20 and then incubated with goat anti-human IgG Fe antibody conjugated with Horse Radish Peroxidase (HRP) for 30 minutes (mins) at room temperature. After washing, the plates were developed with TMB substrate and subsequent stop solution was added to complete the reaction. The signal from each well was read by spectrophotometer at OD 450 nm. As shown in FIG. 1, FIG. 2 and Table 2, most of the tested MerTK antibodies showed comparable binding activities to human or cyno MerTK-his protein to those of benchmark Ab2000-7, M6 and h13B4.v16 antibodies.

TABLE 2
Cross species activity of anti-MerTK
chimeric monoclonal antibodies

EC50 (nM)

| Abs | Human | Cynomolgus |
|---|---|---|
| 10F7D9 | 0.027 | 0.024 |
| 85H8D5 | 0.025 | 0.024 |
| 216G3D6 | 0.046 | 0.047 |
| 247E5A8 | 0.032 | 0.037 |
| 252C12B10 | 0.039 | 0.040 |
| 252H9D5 | 0.052 | 0.150 |
| 254B4D9 | 0.047 | 0.056 |
| 265F11B5 | 0.0097 | 0.017 |
| 276C2D1 | 0.019 | 0.020 |
| 280C6A3 | 0.012 | 0.014 |
| 293C2B7 | 0.017 | 0.020 |
| 300A5A3 | 0.024 | 0.026 |

### Affinity Ranking of Anti-MerTK Chimeric Antibodies by Biacore™

The binding affinity of the MerTK chimeric antibodies to human MerTK-his protein was tested with Biacroe™ T200 using a capture method. Briefly, the antibodies were captured with a Pro-A chip. Two doses (12.5 nM and 50 nM, or 25 nM and 50 nM) of human MerTK-his protein were injected over captured antibody for 180 s at a flow rate of 30 μL/min. The antigen was allowed to dissociate for 420-800 s. Data analysis was carried out using Biacore™ T200 evaluation software. The results are shown in Table 3 below.

TABLE 3
Binding affinity of antibodies to hMerTK protein

hMerTK-His

| Abs | ka (1/Ms) | kd (1/s) | KD (M) |
|---|---|---|---|
| 10F7D9 | 5.542E+5 | 2.380E−3 | 4.295E−9 |
| 85H8D5 | 5.819E+5 | 6.641E−3 | 1.141E−8 |
| 216G3D6 | 1.053E+5 | 8.969E−5 | 8.520E−10 |
| 247E5A8 | 1.298E+5 | 5.959E−4 | 4.589E−9 |
| 252C12B10 | 1.931E+5 | 1.029E−3 | 5.332E−9 |
| 252H9D5 | 2.919E+5 | 2.874E−3 | 9.849E−9 |
| 254B4D9 | 6.551E+4 | 7.825E−4 | 1.194E−8 |
| 265F11B5 | 1.911E+5 | 2.323E−4 | 1.215E−9 |
| 276C2D1 | 1.750E+5 | 2.228E−3 | 1.273E−8 |
| 280C6A3 | 1.792E+5 | 5.526E−4 | 3.084E−9 |
| 300A5A3 | 2.139E+5 | 2.899E−4 | 1.356E−9 |

### Binding to Human MerTK-Overexpressing CHO-K1 Cells

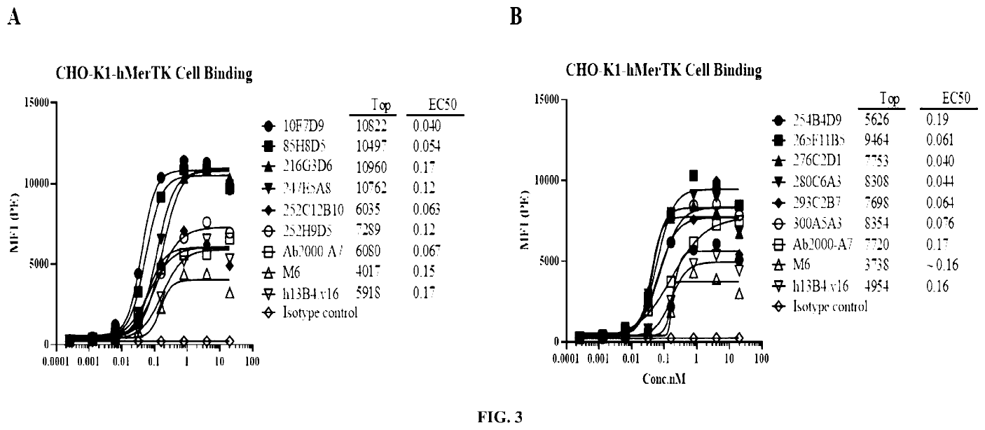

To evaluate the binding property to the antigen expressed on the cells, the chimeric monoclonal antibodies were analyzed for their binding to human MerTK overexpressed on CHO-K1 cells by FACS. Briefly, human MerTK overexpressing CHO-K1 (CHO-K1-MerTK) cells were firstly incubated with 5-fold serially diluted mAbs starting at 20 nM at 4° C. for 1 hour. After washing with FACS buffer, the PE conjugated anti-human IgG-Fc secondary antibody was added to each well and incubated at 4° C., for 30 mins. The mean fluorescence intensity (MFI) of PE were evaluated by QuantAnalyzer 16. As shown in FIG. 3, most of the tested MerTK chimeric antibodies showed higher maximum binding ability (Top) and potency (EC50) to human MerTK expressed on CHOK1 cells than benchmark Ab2000-A7, M6 and h13B4.v16 antibodies.

### Binding to Human MerTK-Overexpressing Tumor Cells

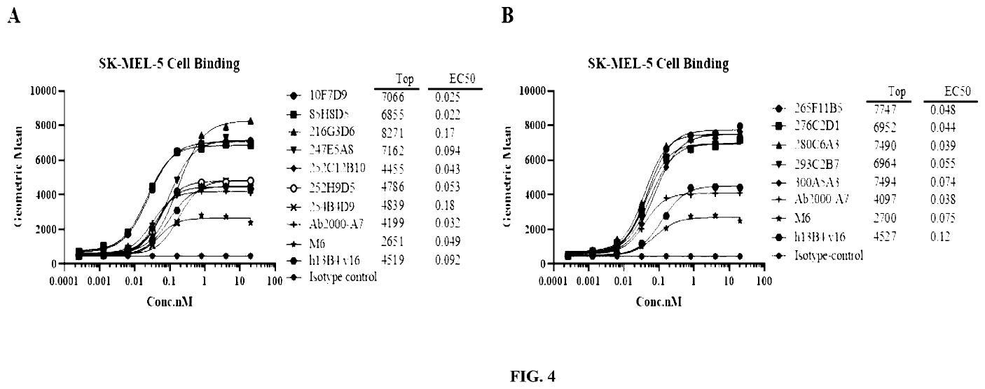

To evaluate the binding properties on MerTK expressed on cells, the chimeric monoclonal antibodies were analyzed for their binding to human melanoma cell line SK-MEL-5 expressing MerTK by FACS analysis. Briefly, SK-MEL-5 cells were firstly incubated with 5-fold serially diluted chimeric mAbs starting at 20 nM at 4° C. for 30 mins. After washing by FACS buffer, the PE conjugated anti-human IgG secondary antibody was incubated with the cell-antibody complex in the wells at 4° C. for 30 mins to detect the antibodies those bound to the cells. The MFI of PE was evaluated by QuantAnalyzer 16. As shown in FIG. 4, most of the tested MerTK chimeric antibodies showed higher maximum binding ability to human MerTK expressed on SK-MEL-5 cells than benchmarkAb2000-A7, M6 and h13B4.v16 antibodies. Of note, some of them showed significantly improved binding potency (EC50) when compared with benchmark antibodies.

### Example 3. Blocking Activities of Anti-MerTK Chimeric Monoclonal Antibodies

### Blockage of MerTK Binding to its Ligand Gas6

The TAM (Tyro3, Axl, and MerTK) family shares the same ligand named Gas6. It is composed of an N-terminal GLA domain, four EGF-like repeats and two laminin G domains at its C-terminal. The N-terminal GLA domain can bind to PtdSer exposed on the plasma membrane of cells in different settings, including apoptosis, immune activation, and coagulation. At the C-terminal, one of the laminin G domains interacts with the Ig-like domains of MerTK to form a heterotetrameric complex and result in downstream signal activation.

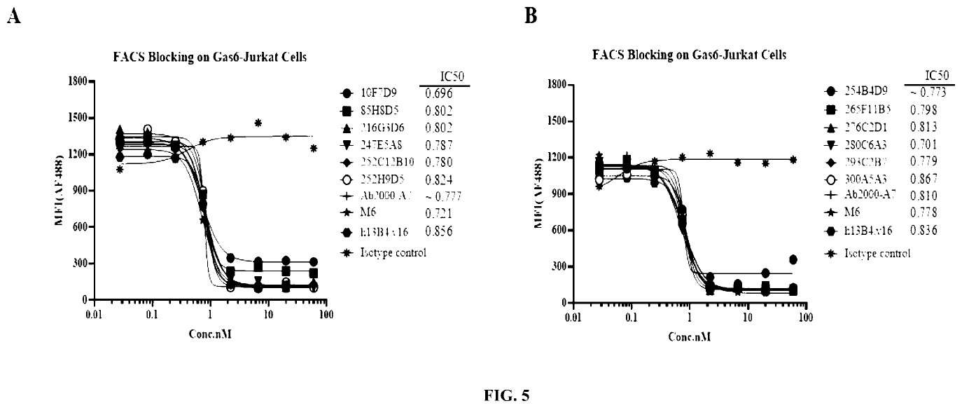

To evaluate the blocking effects of anti-hMerTK mAbs on hMerTK binding to its ligand hGas6, a receptor blocking assay was set-up. Briefly, Jurkat cells engineered to overexpress human Gas6 were added to the 96-well microplates at a density of 5×10⁴ cells per well. Human MerTK-mouse Fe fusion protein (50 μL/well, 1 g/mL) and the MerTK chimeric antibodies with a 3-fold serial dilution starting from 120 nM at a volume of 50 μL were added to the 96-well plates and incubated at 4° C. for 30 mins. After washing with FACS buffer, the plates were incubated with diluted Alex Fluor 488 conjugated goat anti-mouse IgG antibody for 30 mins at 4′C. After washing, the plates were analyzed by QuantAnalyzer 16. As shown in FIG. 5, all the antibodies can efficiently inhibit the binding of human MerTK to human Gas6 expressed on cells.

### Example 4: Functional Properties of Anti-MerTK Antibodies

In this example, efferocytosis assay was carried out to evaluate the inhibiting activity of anti-MerTK antibodies in macrophage-mediated phagocytosis of apoptotic cells in vitro.

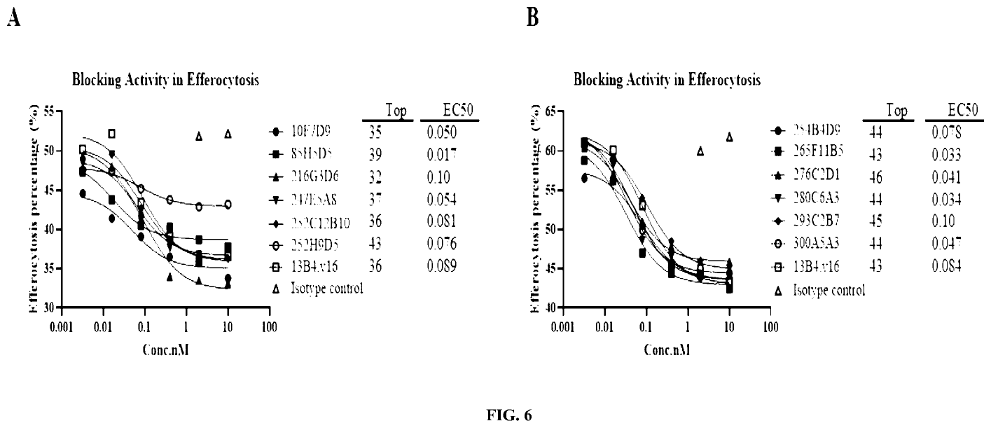

Briefly, Jurkat cells were induced to undergo apoptosis by treatment with 1 μM of Staurosporine for a period of 4 hours. The cells were then washed twice with DPBS and re-suspended in DPBS at a density of 1.0×10⁶ cells/mL. The apoptotic cells were then labeled with 0.5 μM of CFSE in the dark at 37° C. for 5 mins. After labeling, the apoptotic cells were washed with culture medium for 3 times and re-suspended in the culture medium. CD14+ monocytes were isolated with human CD14 microbeads (Miltenyi Biotec) from buffy coat of healthy donors. The CD14+ monocytes were cultured in the presence of 100 ng/ml of M-CSF for 7 days and differentiated into M0 macrophages which showed upregulated MerTK expression. Macrophages were then seeded into the 96-well microplates at a density of 4.0×10⁴ cells/well. Serial dilutions of antibodies were incubated with the macrophages in the 96-well plates for 20 mins. Freshly prepared CFSE-labeled apoptotic Jurkat cells were then added into microwells at a density of 2.0×10⁵ cells/well and co-cultured at 37° C. for 90 mins to allow the phagocytosis of macrophage to apoptotic cells. After the incubation, APC anti-human CD14 antibody was used to label macrophages in the coculture system. Phagocytosis events were quantified by QuantAnalyzer 16. The CD14⁺CFSE⁺ cells represent the macrophages those had phagocytized apoptotic Jurkat cells. The efferocytosis assays demonstrated that anti-MerTK antibodies could inhibit human macrophages from engulfing apoptotic cells. The results in FIG. 6 showed that some of the tested MerTK antibodies displayed more efficient blocking potency in efferocytosis when compared with h13B4.v16.

### Example 5: Humanization of the Chimeric MerTK Antibodies

The variable regions of 10F7D9, 265F11B5 and 254B4D9 chimeric antibodies were selected to perform humanization. Briefly, the amino acid sequences of the VH and VL were aligned with the available database of human Ig gene sequences to identify the overall best-matching human germline Ig gene sequences. For each clone, the CDRs of light chain and heavy chain were grafted onto candidate human germlines. A 3D model was then generated to determine if there were any critical mouse amino acids in the framework region whose replacement to the human amino acid could affect binding and/or CDR conformation. Critical amino acids were selected for backmutation in order to maintain the structure and function of the humanized antibodies. The humanized variable regions of antibody were then fused to the constant region of human IgG1 LALAPG for antibody production.

For the light chain of 10F7D9, the candidate human germline was the IGKV6-21*01 gene. For the heavy chain, the candidate human germline was the IGHV7-4-1*02 gene. In the case of the light chain, L45P, L46W, K48Y and Y86F in the framework regions were involved in back-mutations. In the case of the heavy chain, V2I, V20I, R38K, E46K, S84N, S85N, Y95F and R98T in the framework were involved in back-mutations.

For the light chain of 265F11B5, the candidate human germ line was the IGKV6-21*01 gene. For the heavy chain, the candidate human germline was the IGHV1-18*01 gene. In the case of the light chain, K50Y and F72Y in the framework were involved in back-mutations. In the case of the heavy chain, M481, V68A, M70L, T71A, T72V, Y95F and A97T in the framework were involved in back-mutations.

For the light chain of 254B34D9, the candidate human germline was the IGKV1-33*01 gene, and for the heavy chain the candidate human germline was the IGKV7-4-1*02 or IGKV7-81*01 gene. In the case of the light chain, Y49H, T69R and Y87F in the framework were involved in back-mutations. In the case of the heavy chain, R38K, P38K, E46Q, M72L, Y80F and Y95F in the framework were included as back-mutations. (Tables 4 and 5).

TABLE 4
Humanized antibody sequences (underlining/bold indicates CDR; bold/italic indicates
back mutations)
10F7D9

| | Sequence | SEQ ID NO: |
|---|---|---|
| VH mouse | QIQLVQSGPELKKPGETVKISCKASGYTFTNYGMNWVKQAPGKGLK | 1 |
| | WMGWINTYTGEPTYADDFKGRFAFSLETSASTAYLQINNLKNEDLA | |
| | TYFCATYGHYAWFAYWGQGTLVTVSA | |
| HU-VH1-0 | QVQLVQSGSELKKPGASVKVSCKASGYTFTNYGMNWVRQAPGQGLE | 82 |
| | WMGWINTYTGEPTYADDFKGRFVFSLDTSVSTAYLQISSLKAEDTA | |
| | VYYCARYGHYAWFAYWGQGTLVTVSS | |
| HU-VH1-1 | QIQLVQSGSELKKPGASVKVSCKASGYTFTNYGMNWVRQAPGQGLE | 83 |
| | WMGWINTYTGEPTYADDFKGRFVFSLDTSVSTAYLQISSLKAEDTA | |
| | VYYCATYGHYAWFAYWGQGTLVTVSS | |
| HU-VH1-2 | QIQLVQSGSELKKPGASVKVSCKASGYTFTNYGMNWVRQAPGQGLE | 84 |
| | WMGWINTYTGEPTYADDFKGRFVFSLDTSVSTAYLQISSLKAEDTA | |
| | VYFCATYGHYAWFAYWGQGTLVTVSS | |
| HU-VH1-3 | QIQLVQSGSELKKPGASVKVSCKASGYTFTNYGMNWVRQAPGQGLK | 85 |
| | WMGWINTYTGEPTYADDFKGRFVFSLDTSVSTAYLQISSLKAEDTA | |
| | VYFCATYGHYAWFAYWGQGTLVTVSS | |
| HU-VH1-4 | QIQLVQSGSELKKPGASVKVSCKASGYTFTNYGMNWVKQAPGQGLK | 86 |
| | WMGWINTYTGEPTYADDFKGRFVFSLDTSVSTAYLQISSLKAEDTA | |
| | VYFCATYGHYAWFAYWGQGTLVTVSS | |
| HU-VH1-5 | QVQLVQSGSELKKPGASVKISCKASGYTFTNYGMNWVRQAPGQGLE | 87 |
| | WMGWINTYTGEPTYADDFKGRFVFSLDTSVSTAYLQINNLKAEDTA | |
| | VYFCATYGHYAWFAYWGQGTLVTVSS | |
| IGHV7-4- | QVQLVQSGSELKKPGASVKVSCKASGYTFTSYAMNWVRQAPGQGLE | 88 |
| 1*02 | WMGWINTNTGNPTYAQGFTGRFVFSLDTSVSTAYLQISSLKAEDTA | |
| | VYYCAR | |
| VL mouse | QIVLSQSPAILSASPGEKVTMTCRASSSVSYMHWYQQKSGSSPKPW | 2 |
| | IYATSNLASGVPARFSGSGSGTSYSLTISRVEAEDAATYFCQQWGT | |
| | KPLTFGAGTKLELK | |
| HU-VL1-0 | EIVLTQSPDFQSVTPKEKVTITCRASSSVSYMHWYQQKPDQSPKLL | 89 |
| | IKATSNLASGVPSRFSGSGSGTDFTLTINSLEAEDAATYYCQQWGT | |
| | KPLTFGQGTKLEIK | |
| HU-VL1-1 | EIVLTQSPDFQSVTPKEKVTITCRASSSVSYMHWYQQKPDQSPKPW | 90 |
| | IKATSNLASGVPSRFSGSGSGTDFTLTINSLEAEDAATYYCQQWGT | |
| | KPLTFGQGTKLEIK | |
| HU-VL1-2 | EIVLTQSPDFQSVTPKEKVTITCRASSSVSYMHWYQQKPDQSPKPW | 91 |
| | IYATSNLASGVPSRFSGSGSGTDFTLTINSLEAEDAATYFCQQWGT | |
| | KPLTFGQGTKLEIK | |
| IGKV6-21*01 | EIVLTQSPDFQSVTPKEKVTITCRASQSIGSSLHWYQQKPDQSPKL | 92 |
| | LIKYASQSFSGVPSRFSGSGSGTDFTLTINSLEAEDAATYYCHQSS | |
| | SLP | |

265F11B5

| | Sequence | SEQ ID NO: |
|---|---|---|
| VH mouse | QVQLQQSGPELVKPGASVRLSCKASGYTFTTYDINWVKQRPGLGLE | 15 |
| | WIGWIYPRDGYTKYNEKFKGRATLAVDTSSSTAYMELHSLTSEDSA | |
| | VYFCTRGYYDNLYYFDYWGQGTTLTVSS | |
| HU-VH1-0 | QVQLVQSGAEVKKPGASVKVSCKASGYTFTTYDINWVRQAPGQGLE | 93 |
| | WMGWIYPRDGYTKYNEKFKGRVTMTTDTSTSTAYMELRSLRSDDTA | |
| | VYYCARGYYDNLYYFDYWGQGTTVTVSS | |
| HU-VH1-1 | QVQLVQSGAEVKKPGASVKVSCKASGYTFTTYDINWVRQAPGQGLE | 94 |
| | WMGWIYPRDGYTKYNEKFKGRVTMTTDTSTSTAYMELRSLRSDDTA | |
| | VYYCTRGYYDNLYYFDYWGQGTTVTVSS | |
| HU-VH1-2 | QVQLVQSGAEVKKPGASVKVSCKASGYTFTTYDINWVRQAPGQGLE | 95 |
| | WIGWIYPRDGYTKYNEKFKGRATMTTDTSTSTAYMELRSLRSDDTA | |
| | VYFCTRGYYDNLYYFDYWGQGTTVTVSS | |
| HU-VH1-3 | QVQLVQSGAEVKKPGASVKVSCKASGYTFTTYDINWVRQAPGQGLE | 96 |
| | WIGWIYPRDGYTKYNEKFKGRATLAVDTSTSTAYMELRSLRSDDTA | |
| | VYFCTRGYYDNLYYFDYWGQGTTVTVSS | |
| IGHV1-18*01 | QVQLVQSGAEVKKPGASVKVSCKASGYTFTSYGISWVRQAPGQGLE | 97 |
| | WMGWISAYNGNTNYAQKLQGRVTMTTDTSTSTAYMELRSLRSDDTA | |
| | VYYCAR | |
| VL mouse | EIVLTQSPTTVAASPGEKITITCSASSIISSNYFHWYQQRPGFSPK | 16 |
| | LLIYRTSNLASGVPARFSGSGSGTSYSLTIGTMEAEDVATYYCQQG | |
| | RSVPLTFGAGTKLELK | |
| HU-VL1-0 | EIVLTQSPDFQSVTPKEKVTITCSASSIISSNYFHWYQQKPDQSPK | 98 |
| | LLIKRTSNLASGVPSRFSGSGSGTDFTLTINSLEAEDAATYYCQQG | |
| | RSVPLTFGQGTKLEIK | |
| HU-VL1-1 | EIVLTQSPDFQSVTPKEKVTITCSASSIISSNYFHWYQQKPDQSPK | 99 |
| | LLIKRTSNLASGVPSRFSGSGSGTDYTLTINSLEAEDAATYYCQQG | |
| | RSVPLTFGQGTKLEIK | |
| HU-VL1-2 | EIVLTQSPDFQSVTPKEKVTITCSASSIISSNYFHWYQQKPDQSPK | 100 |
| | LLIYRTSNLASGVPSRFSGSGSGTDYTLTINSLEAEDAATYYCQQG | |
| | RSVPLTFGQGTKLEIK | |
| IGKV6-21*01 | EIVLTQSPDFQSVTPKEKVTITCRASQSIGSSLHWYQQKPDQSPKL | 101 |
| | LIKYASQSFSGVPSRFSGSGSGTDFTLTINSLEAEDAATYYCHQSS | |
| | SLP | |

254B4D9

| | Sequence | SEQ ID NO: |
|---|---|---|
| VH mouse | QIQLVQSGPELKKPGETVKISCKASGYTFTAYGMSWVKQAPGKGLQ | 13 |
| | WMGWINTYSGVPTYTDDFKGRFAFSLEASASTAFLQINNLKNEDTS | |
| | TYFCARRAAVVAKYWYFDVWGTGTTVTVSS | |
| HU-VH1 | QVQLVQSGSELKKPGASVKVSCKASGYTFTAYGMSWVKQAPGQGLQ | 102 |
| | WMGWINTYSGVPTYTDDFKGRFVFSLDTSVSTAFLQISSLKAEDTA | |
| | VYFCARRAAVVAKYWYFDVWGQGTTVTVSS | |
| IGHV7-4- | QVQLVQSGSELKKPGASVKVSCKASGYTFTSYAMNWVRQAPGQGLE | 103 |
| 1*02 | WMGWINTNTGNPTYAQGFTGRFVFSLDTSVSTAYLQISSLKAEDTA | |
| | VYYCAR | |
| HU-VH2 | QVQLVQSGHEVKQPGASVKVSCKASGYSFTAYGMSWVKQAPGQGLQ | 104 |
| | WMGWINTYSGVPTYTDDFKGRFVFSLDTSASTAFLQISSLKAEDMA | |
| | MYFCARRAAVVAKYWYFDVWGQGTTVTVSS | |
| IGHV7-81*01 | QVQLVQSGHEVKQPGASVKVSCKASGYSFTTYGMNWVPQAPGQGLE | 105 |
| | WMGWFNTYTGNPTYAQGFTGRFVFSMDTSASTAYLQISSLKAEDMA | |
| | MYYCAR | |
| VL mouse | DIQMTQSPSSLFASLGGKVIITCKSSQDINKRISWYQHKPGKGPRL | 14 |
| | LIHSTSSLQPGIPLRFSGSGSGRDYSFSISNLEPEDIATYFCLQYD | |
| | NLLTFGAGTKLELK | |
| HU-VL1 | DIQMTQSPSSLSASVGDRVTITCKSSQDINKRISWYQQKPGKAPKL | 106 |
| | LIHSTSSLQPGVPSRFSGSGSGRDFTFTISSLQPEDIATYFCLQYD | |
| | NLLTFGQGTKLEIK | |
| IGHV1-33*01 | DIQMTQSPSSLSASVGDRVTITCQASQDISNYLNWYQQKPGKAPKL | 107 |
| | LIYDASNLETGVPSRFSGSGSGTDFTFTISSLQPEDIATYYCQQYD | |
| | NLP | |

TABLE 5
Pairing of VH and VL for humanized antibodies

| | HU-VL1-0 | HU-VL1-1 | HU-VL1-2 | VL |
|---|---|---|---|---|
| 10F7D9 | | | | |
| HU-VH1-0 | Hu-1 | Hu-7 | Hu-13 | |
| HU-VH1-1 | Hu-2 | Hu-8 | Hu-14 | |
| HU-VH1-2 | Hu-3 | Hu-9 | Hu-15 | |
| HU-VH1-3 | Hu-4 | Hu-10 | Hu-16 | |
| HU-VH1-4 | Hu-5 | Hu-11 | Hu-17 | |
| HU-VH1-5 | Hu-6 | Hu-12 | Hu-18 | |
| VH | | | | Chimeric |
| 265F11B5 | | | | |
| HU-VH1-0 | Hu-1 | Hu-5 | Hu-9 | |
| HU-VH1-1 | Hu-2 | Hu-6 | Hu-10 | |
| HU-VH1-2 | Hu-3 | Hu-7 | Hu-11 | |
| HU-VH1-3 | Hu-4 | Hu-8 | Hu-12 | |
| VH | | | | Chimeric |

| 254B4D9 | HU-VL1 | VL |
|---|---|---|
| HU-VH1 | Hu-1 | |
| HU-VH2 | Hu-2 | |
| VH | | Chimeric |

### Example 6: Antigen Binding Properties of the Humanized Antibodies

### Binding to Recombinant Human MerTK

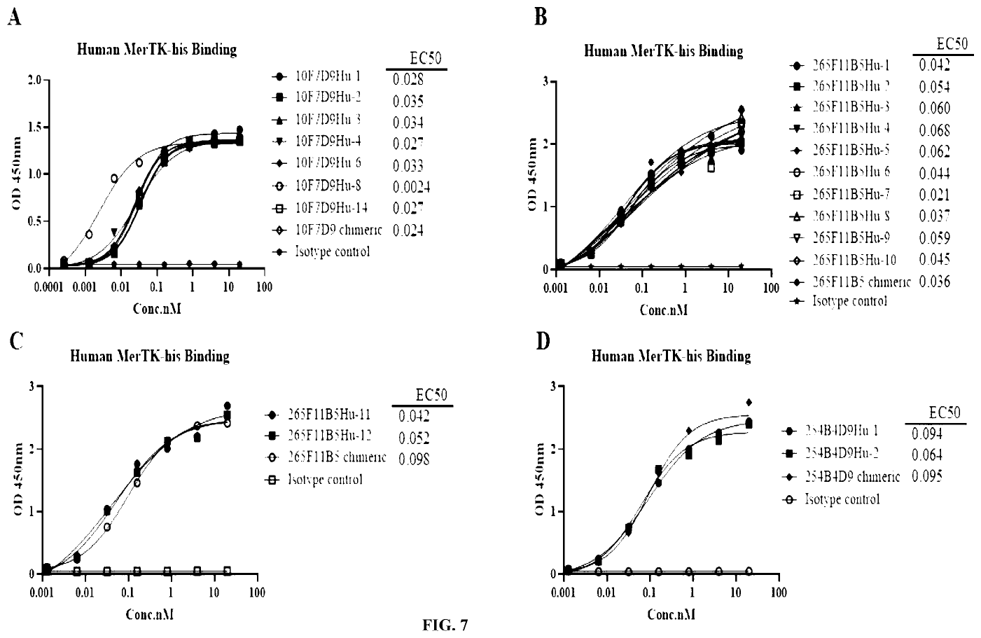

To evaluate the antigen binding activity, the humanized antibodies were subjected to ELISA test as described before. As shown in FIG. 7, the humanized antibodies showed comparable binding efficacy to human MerTK to their parental chimeric antibodies.

### Affinity Ranking of Humanized Antibodies by Biacore™

To explore whether the humanized antibodies could maintain their binding kinetics, affinity ranking were performed with Biacore™. The antibodies were captured with a Protein A chips. Human MerTK-his protein at 50 nM was injected over captured antibodies for 180 s at a flow rate of 30 μL/min. The antigen was allowed to dissociate for 600 s. The experiment was carried out on a Biacore™ 8K. Data analysis was carried out using Biacore™ 8K evaluation software. The results are shown in Table 6, 265F11B5Hu-4, 265F11B5Hu-7, 265F11B5Hu-8, 254B4D9Hu-1, and 254B4D9Hu-2 showed comparable affinity to their chimeric antibodies.

TABLE 6
Affinity ranking results of humanized antibodies

Human MerTK His

| | ka(1/Ms) | Kd(1/s) | KD(M) |
|---|---|---|---|
| 265F11B5Hu-1 | 2.01E+05 | 2.32E−04 | 1.15E−09 |
| 265F11B5Hu-2 | 1.97E+05 | 2.50E−04 | 1.27E−09 |
| 265F11B5Hu-3 | 1.99E+05 | 2.04E−04 | 1.02E−09 |
| 265F11B5Hu-4 | 2.01E+05 | 1.72E−04 | 8.56E−10 |
| 265F11B5Hu-5 | 2.00E+05 | 2.32E−04 | 1.16E−09 |
| 265F11B5Hu-6 | 1.99E+05 | 2.78E−04 | 1.40E−09 |
| 265F11B5Hu-7 | 2.16E+05 | 1.55E−04 | 7.19E−10 |
| 265F11B5Hu-8 | 2.27E+05 | 3.95E−05 | 1.74E−10 |
| 265F11B5Hu-9 | 2.04E+05 | 4.43E−04 | 2.17E−09 |
| 265F11B5Hu-10 | 1.84E+05 | 4.63E−04 | 2.52E−09 |
| 265F11B5Hu-11 | 1.95E+05 | 3.78E−04 | 1.94E−09 |
| 265F11B5Hu-12 | 1.89E+05 | 3.10E−04 | 1.64E−09 |
| 265F11B5-chimeric | 1.99E+05 | 7.78E−05 | 3.92E−10 |
| 254B4D9Hu-1 | 2.17E+05 | 4.75E−04 | 2.19E−09 |
| 254B4D9Hu-2 | 5.41E+04 | 5.92E−04 | 1.09E−08 |
| 254B4D9-chimeric | 2.25E+04 | 7.85E−04 | 3.49E−08 |

### Binding to Human MerTK Overexpressed CHO-K1 Cells

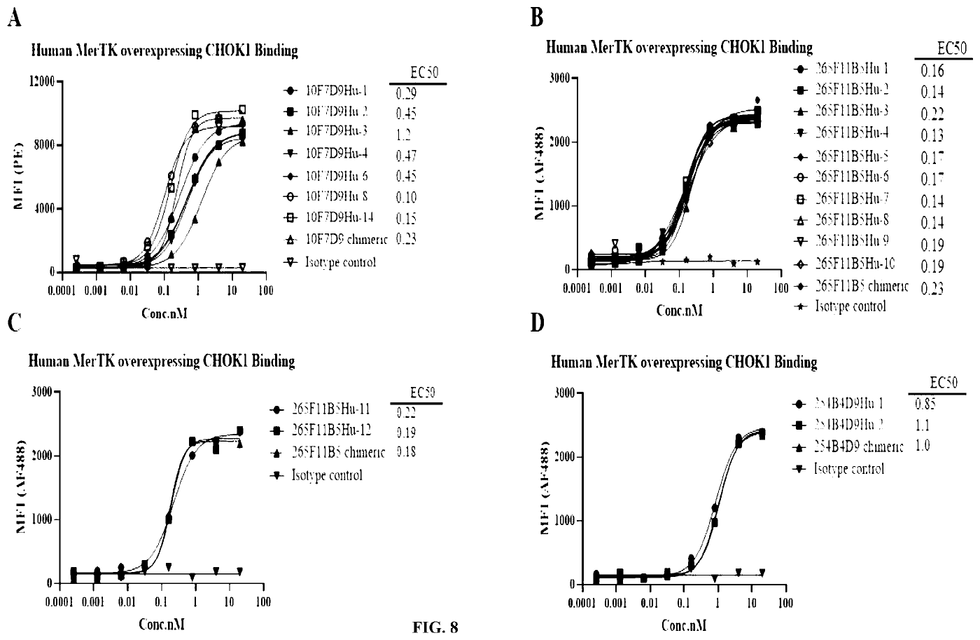

To evaluate the binding property to the antigen expressed on the cells, the humanized antibodies were analyzed by FACS as described before. As shown in FIG. 8, the humanized antibodies of 265F11B5 and 254B4D9 showed comparable cell binding efficacy to their chimeric antibodies. In the meanwhile, some of the humanized antibodies of 10F7D9 showed comparable cell binding efficacy to their chimeric antibody.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or antigen-binding fragment thereof which has specificity to the human Mer proto-oncogene tyrosine-protein kinase (MerTK) protein and comprises a heavy chain variable region (VH) comprising a VH CDR1, a VH CDR2 and a VH CDR3, and a light chain variable region (VL) comprising a VL CDR1, a VL CDR2, and a VL CDR3, wherein the VH CDR1, VH CDR2, VH CDR3, VL CDR1, VL CDR2 and VL CDR3 comprise, respectively, the amino acid sequences of
SEQ ID NO: 25-30;
SEQ ID NO: 59-64;
SEQ ID NO: 65-70;
SEQ ID NO: 31-33, 28-29, 34;
SEQ ID NO: 35-40;
SEQ ID NO: 41-46;
SEQ ID NO: 47-52;
SEQ ID NO: 53-58;
SEQ ID NO: 71, 66, 72, 73, 69, 74;
SEQ ID NO: 65, 75, 67, 76, 69, 77;
SEQ ID NO: 71, 78-79, 73, 69, 74; or
SEQ ID NO: 65-66, 80-81, 69-70.

2. The antibody or antigen-binding fragment thereof of claim 1, wherein:
the VH CDR1 comprises the amino acid sequence of SEQ ID NO: 25;
the VH CDR2 comprises an amino acid sequence of SEQ ID NO: 26;
the VH CDR3 comprises the amino acid sequence of SEQ ID NO: 27;
the VL CDR1 comprises the amino acid sequence of SEQ ID NO: 28;
the VL CDR2 comprises the amino acid sequence of SEQ ID NO: 29; and
the VL CDR3 comprises the amino acid sequence of SEQ ID NO: 30.

3. The antibody or antigen-binding fragment thereof of claim 2, wherein the VH comprises an amino acid sequence selected from the group consisting of SEQ ID NO: 1, and 82-87, and the VL comprises an amino acid sequence selected from the group consisting of SEQ ID NO: 2 and 89-91.

4. The antibody or antigen-binding fragment thereof of claim 2, wherein the VH comprises the amino acid sequence of SEQ ID NO: 83, and the VL comprises the amino acid sequence of SEQ ID NO: 90.

5. The antibody or antigen-binding fragment thereof of claim 2, wherein the VH comprises the amino acid sequence of SEQ ID NO: 83, and the VL comprises the amino acid sequence of SEQ ID NO: 91.

6. The antibody or antigen-binding fragment thereof of claim 1, wherein:
the VH CDR1 comprises the amino acid sequence of SEQ ID NO: 59;
the VH CDR2 comprises an amino acid sequence of SEQ ID NO: 60;
the VH CDR3 comprises the amino acid sequence of SEQ ID NO: 61;
the VL CDR1 comprises the amino acid sequence of SEQ ID NO: 62;
the VL CDR2 comprises the amino acid sequence of SEQ ID NO: 63; and
the VL CDR3 comprises the amino acid sequence of SEQ ID NO: 64.

7. The antibody or antigen-binding fragment thereof of claim 6, wherein the VH comprises an amino acid sequence selected from the group consisting of SEQ ID NO: 13, 102 and 104, and the VL comprises an amino acid sequence selected from the group consisting of SEQ ID NO: 14 and 106.

8. The antibody or antigen-binding fragment thereof of claim 6, wherein the VH comprises the amino acid sequence of SEQ ID NO: 102, and the VL comprises the amino acid sequence of SEQ ID NO: 106.

9. The antibody or antigen-binding fragment thereof of claim 6, wherein the VH comprises the amino acid sequence of SEQ ID NO: 104, and the VL comprises the amino acid sequence of SEQ ID NO: 106.

10. The antibody or antigen-binding fragment thereof of claim 1, wherein:
the VH CDR1 comprises the amino acid sequence of SEQ ID NO: 65;
the VH CDR2 comprises an amino acid sequence of SEQ ID NO: 66;
the VH CDR3 comprises the amino acid sequence of SEQ ID NO: 67;
the VL CDR1 comprises the amino acid sequence of SEQ ID NO: 68;
the VL CDR2 comprises the amino acid sequence of SEQ ID NO: 69; and
the VL CDR3 comprises the amino acid sequence of SEQ ID NO: 70.

11. The antibody or antigen-binding fragment thereof of claim 10, wherein the VH comprises an amino acid sequence selected from the group consisting of SEQ ID NO: 15 and 93-96, and the VL comprises an amino acid sequence selected from the group consisting of SEQ ID NO: 16 and 98-100.

12. The antibody or antigen-binding fragment thereof of claim 10, wherein the VH comprises the amino acid sequence of SEQ ID NO: 95, and the VL comprises the amino acid sequence of SEQ ID NO: 99.

13. The antibody or antigen-binding fragment thereof of claim 10, wherein the VH comprises the amino acid sequence of SEQ ID NO: 96, and the VL comprises the amino acid sequence of SEQ ID NO: 99.

14. (canceled)

15. A multispecific antibody comprising an antigen-binding fragment of claim 1 and one or more antibody or antigen-binding fragment having binding specificity to a target antigen that is not MerTK.

16. A chimeric antigen receptor (CAR) comprising an antigen-binding fragment of claim 1, a transmembrane domain, a costimulatory domain, and a CD3ξ intracellular domain.

17. One or more polynucleotide(s) encoding the antibody or antigen-binding fragment thereof of claim 1.

18-19. (canceled)

20. A cell comprising the polynucleotide(s) of claim 17.

21. (canceled)

22. A method of treating cancer or an inflammatory condition in a patient in need thereof, comprising administering to the patient an effective amount of the antibody or antigen-binding fragment thereof of claim 1.

23. (canceled)

24. The method of claim 22, wherein the cancer is a solid tumor.

25. (canceled)

26. The method of claim 22, wherein the inflammatory condition is selected from the group consisting of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, and ulcerative colitis.
